SEQUENCE LISTING (6 sequences)

SEQ ID NO: 1 - DNA, 20 bp, Artificial Sequence
  Primer
gatgccctgaggtactgttc

SEQ ID NO: 2 - DNA, 20 bp, Artificial Sequence
  Primer
acctccttgctcactctgtc

SEQ ID NO: 3 - DNA, 20 bp, Artificial Sequence
  Primer
atagcttgcgtctggatagg

SEQ ID NO: 4 - DNA, 20 bp, Artificial Sequence
  Primer
tgcgtcctctcatgtaaaaa

SEQ ID NO: 5 - DNA, 19 bp, Artificial Sequence
  Primer
caacatcttcgtggacctg

SEQ ID NO: 6 - DNA, 18 bp, Artificial Sequence
  Primer
aaggcgttgagatgcagt

(19) United States
(12) Patent Application Publication
(10) Pub. No.: US 2018/0066288 A1
(43) Pub. Date: Mar. 8, 2018

(54) PRODUCING AND ALTERING MICROBIAL FERMENTATION PRODUCTS USING NON-COMMONLY USED LIGNOCELLULOSIC HYDROLYSATES

(71) Applicant: Kuehnle AgroSystems, Inc., Honolulu, HI (US)

(72) Inventors: Norie Anne B. Nolasco, Honolulu, HI (US); Amit Vasavada, Poway, CA (US); Adelheid R. Kuehnle, Honolulu, HI (US); Robert J. Schurr, Honolulu, HI (US)

(21) Appl. No.: 15/669,715

(22) Filed: Aug. 4, 2017

Related U.S. Application Data
(60) Provisional application No. 62/371,492, filed on Aug. 5, 2016.

(51) Int. Cl. C12P 7/06; C12P 19/14; C12N 1/12; C12P 39/00; C08H 8/00

(52) U.S. Cl. CPC C12P 7/065; C12P 19/14; C08H 8/00; C12P 39/00; C12N 1/12

(57) ABSTRACT

The invention pertains to a method for synthesizing a product of interest by culturing a microbe that produces the product of interest, the method comprising culturing the microbe in a culture medium, wherein the culture medium is produced by a method comprising the steps of:
a) providing a lignocellulosic biomass,
b) hydrolyzing the lignocellulosic biomass to produce a lignocellulosic hydrolysate comprising a simplified sugar produced from at least a portion of the lignocellulosic compound,
c) optionally, treating a portion of the lignocellulosic hydrolysate to convert a portion of the lignocellulosic compound and/or the simplified sugar to a non-sugar agent;
d) optionally, mixing the treated portion of the lignocellulosic hydrolysate, if produced, with the untreated portion of the lignocellulosic hydrolysate,
e) producing a culture medium comprising the lignocellulosic hydrolysate obtained after step b) or comprising the mixture obtained after steps c) and d).

This work was supported in part by USDA Award 2011-10006-30377 Integrated Biorefinery at the Domtar Plymouth, N.C. Pulp Mill, sub-award to Kuehnle Agro Systems.

### FIELD OF THE INVENTION

The present invention relates to microbial fermentation methods for synthesizing useful products resulting from the incorporation of non-commonly used lignocellulosic derivatives into culture medium. In one embodiment, the present invention relates to fermentation methods employing heterotrophic and/or mixotrophic culturing of microorganisms with softwood or hardwood lignocellulosic simplified sugar in the presence of a non-sugar agent that is a wood-derived lignocellulose hydrolysis process or wood-derived organic acid solution.

### BACKGROUND OF THE INVENTION

Global impetus is growing to provide alternatives to using fossil fuels and diminished non-renewable resources as sources of products that are used in large quantities. Such products can be used for production agriculture and aquaculture, health care, and industrial applications, and can span from compounds or ingredients for feed, foods, beverages, dietary supplements, crop protection, and personal care to raw materials for chemical manufacturing. Non-limiting examples of components that comprise the products include proteins, lipids, pigments, industrial polymers and recombinant molecules. Manufacturing these products using suitable microorganisms, such as microalgae, to replace unsustainable or problematic products or ingredients currently used in the marketplace and to do so using economic inputs for production is valuable.

The use of microalgae as biofactories to generate massive volumes of renewable biomass and bioproducts at competitive prices requires availability of abundant and relatively inexpensive feedstocks for fermentative bioconversion (heterotrophic or mixotrophic). Aerobic fermentation by heterotrophic algae is performed using generally similar fermentor tanks and operations as seen for other microorganisms in industrial fermentation facilities. Fermentation is considered the most economical and scalable method of algae production. In such fermentation, light can be used for mixotrophic growth by facultative heterotrophic microalgae using fixed carbon as well photosynthesis as a carbon source. Fermentation can also proceed in darkness using fixed carbon with no photosynthesis by facultative or obligate heterotrophic microalgae.

Plant-based cellulosic sugars are increasingly attractive sources for feedstocks for use in microbial fermentation. These are generally from agricultural wastes or residues that remain after harvest or processing, purposefully grown energy grasses or invasive grasses, and low cost forestry-based biomass. Some examples of agricultural wastes include corn stover, soybean stover, wheat straw, barley straw, rice straw, oat straw, oat hulls, canola straw, and sugar processing residues such as bagasse and beet pulp. Some examples of grasses include switch grass, sweet sorghum, Miscanthus, and cordgrass. Forestry-based biomass includes underutilized wood (hardwood and softwood) and forest residues (bark, etc.); purposefully grown energy feedstocks include certain short-rotation hardwood coppice crops, such as willow, poplar, robina, and eucalyptus.

Underutilized woody biomass can be obtained from the pulp and paper industry that processes wood for various uses, for example, printing and writing paper grades, various coated and uncoated specialties paper grades, tissue and toweling products, paperboard, medical packaging, absorbent and air laid non-woven products (such as diapers, hygiene, incontinence products), textile fibers, film, and sawn timber. These products utilize many types of wood that may comprise but are not limited to Northern Softwood (for example Lodgepole Pine, White/Engelmann Spruce, Jack Pine, Sitka Spruce, Norway Spruce, and Black Spruce); Northern Hardwood (for example Maple, Birch, Poplar); Southern Softwood (for example Loblolly Pine, Shortleaf Pine); Southern Hardwood (for example Oak, Maple and Poplar). The other wood-based biomass in the supply chain comprises but is not limited to debarking residues, chip screening residues, knots and pulp fibers. The associated mills can be of various types and can include chemical pulp mills (such as sulfate mills and sulfite mills) and chemical-mechanical pulp mills (such as TMP and CTMP mills).

Unlike agricultural wastes or energy grasses such as stover and switchgrass, which can be highly seasonal for a specific geography, and therefore, pose serious logistical challenges inherent in shifting among feedstocks for operation of a biorefinery, large quantities of wood-based biomass are available all year round at a given location. Advantageously, large quantities of wood that can be transformed into cellulosic feedstock can support on-site, year-round, low cost industrial scaling to generate microbial biomass required annually for high-volume applications. This in turn can displace significant quantities of petroleum-derived products and reduce reliance on environmentally unsound practices.

Pulp and paper mills have access to an abundance of low cost wood. Reduction to its component wood sugars such as by hydrolytic treatment may make it a potential feedstock for production of valuable algal biomass as part of an integrated biorefinery. Much higher resistance to degradation of biomass from wood, compared to biomass from the grasses and agricultural wastes, means the upstream processing pre-treatments and conditions as well as the downstream saccharification processes and conditions are necessarily different. While the use of cellulosic sugars and cellulosic hydrolysates from some non-wood sources is viable for supporting microalgal biomass production, use of wood-derived lignocellulosic hydrolysates for algal production has not been achieved until the present invention.

Wood cellulosic material and hemicellulosic material can be pre-treated and hydrolyzed by several processes known in the art. Non-limiting examples for producing wood-based sugar can comprise a biomass pre-treatment, which mainly fractionates biomass, followed by hydrolysis in which some fractions of biomass are converted into sugar. The most common lignocellulosic biomass pre-treatment techniques include: (a) physical (e.g., chipping, grinding, milling, etc.); (b) biological; (c) chemical (e.g., using acids, alkalines, solvents, ozone, peroxide, etc.); and (d) physico-chemical processes (e.g., steam explosion, hot water extraction, ammonia fiber extraction, etc.). These processes yield hydrolysates comprised of monosaccharides—simplified hexose and pentose sugars such as of glucan (C6), xylan (C5), arabinan (C5), mannan (C6), and galactan (C6)—along with other wood-derived non-sugar constituents, co-products/by-products and process residuals that carry over from prior pre-treatment and treatment steps.

These other constituents, co-products/by-products, or residuals are considered impurities and are recognized as inhibitors of microbial growth. One potential shortcoming is the resulting impurities and inhibitors in the wood hydrolysates that may be incompatible with cultivation of microbes, specifically, microalgae.

To overcome this shortcoming, sugars from lignocellulosic hydrolysates can be further processed (i.e., detoxified or conditioned) into purified sugars to yield monosaccharide feedstocks devoid of the toxic impurities in unpurified hydrolysate to support microbial growth and bioconversion activities. For example, U.S. Pat. No. 8,889,402 describes cultivating heterotrophically in the dark a genetically engineered Chlorella protothecoides on pure carbon feedstock. U.S. Pat. No. 7,063,957 describes cultivating Chlorella zofingiensis grown on glucose and producing pigment. U.S. Pat. No. 7,674,609 discloses cultivating Crypthecodinium cohnii on reagent grade glucose and organic acid. U.S. Pat. No. 8,889,402 discloses that Scenedesmus armatus and Navicula pelliculosa grow better when pure carbon sources are added sequentially than when added together at the outset. Other work describes sugar uptake inhibition or transporter repression in mixed fixed carbon feedstocks. However, utilization of reagent grade components cannot predict performance of unpurified components in a composite solution. Also, the nature of wood lignocellulosic hydrolysate is such that the various carbon sources are added together at the outset as they are not purified carbon streams.

Unpurified wood-derived lignocellulosic hydrolysates would be more convenient feedstock for microbial conversions, to minimize equipment, time, and energy inputs required for the further fractionation, and purification steps into purified components. While researchers have suggested that cellulose hydrolysis solutions can be a low cost substitute for glucose as a carbon source in the fermentation process, they have also recognized that wood lignocellulosic hydrolysis is difficult and costly. Therefore, having favorably altered profiles of target products can increase the value of the algal product and therefore enable greater economic returns.

Forest based companies may also integrate options at mill sites to produce organic acids such as acetic acid from a partial stream of lignocellulosic hydrolysate that can further serve as preferred fermentation feedstock for certain microbes. A mill may also choose to condition a partial stream of hydrolysate that can serve as preferred fermentation feedstock for certain microbes, for example, with use of a metal salt as described in US Patent Application Publication No. 20110318798.

There are discriminating characteristics between hardwoods and softwoods, just as there are characteristic differences between wood and the herbaceous grasses or sugar crop processing residues. Not all plant cell walls have the same cellulose, hemicellulose and lignin contents and compositions. Consequently, these differences are expected to give rise to differences in lignocellulosic hydrolysates.

It is well known that the composition and structure of the softwood and hardwood hemicelluloses differ, with the major class of hardwood hemicelluloses being the glucuronoxylans. This xylan is O-acetyl-(4-O-methylglucurono)-b-D-xylan, with the xylan backbone having glucuronic acid substituents. The content of glucuronoxylan in hardwoods is typically between 15 and 30% by weight. In some birches xylan content can reach as high as 35%. Also, unlike softwoods, partial acetylation may occur on the 2 or 3 positions of the xylose backbone to yield, for example, seven acetyl residues per ten xylose units. Xylosidic bonds between xylose units are easily hydrolyzed by acids, in contrast to linkages between uronic acid groups and xylose that are very resistant. The acetyl groups are easily cleaved by alkali. Hardwoods also usually contain small amounts (2-5%) of glucomannan. It is composed of β-D-glucopyranose and β-D-mannopyranose linked by (1→4) bonds and the glucose to mannose residues are generally in the ratio of 1:2. Mannosidic bonds between mannose units are more rapidly hydrolyzed than the corresponding glucosidic bonds and glucomannan is easily depolymerized under acidic conditions.

The major class of softwood hemicelluloses is O-acetyl-galactoglucomannan, with the glucose to mannose ratio of about 1:3, and the ratio of galactose to glucose varying from 1:1 to 1:10. Softwood xylan is an arabino-(4-O-methylglucurono)xylan. In contrast to hardwood, the softwood xylan does not contain acetyl groups and is more highly branched and more acidic than the hardwood xylan. These side chains can be removed under mild acidic conditions in which the main xylose chain remains intact. The arabinose and uronic acid substituents stabilize the xylan chain against alkali-catalyzed degradation. The lignin fraction of softwoods such as pine is generally considerably more than in temperate hardwoods, although this is not always the case, and can be nearly double compared to corn stover. While most lignin can be filtered out, their presence in process hydrolysates may cause issues as seen in ethanol fermentations.

Preferably, a hydrolysate feedstock would be suitable for use by microalgae that are capable of complete utilization of the C5 and C6 sugars for maximum biomass yield. Many microalgae strains appear unable to utilize pentose and hexose during fermentation. Some species utilize xylose or other pentose sugars with increased productivity only when grown in the presence of light. Difficulty in a cell's utilization of the cellulose and hemicellulose-derived sugars has been addressed for some algae using genetic engineering, for example, for uptake or modification of polysaccharides as disclosed in U.S. Pat. No. 8,889,402 and U.S. Pat. No. 8,592,188; of cellodextrin as disclosed in U.S. Pat. No. 8,431,360; or of pentose as disclosed in U.S. Pat. No. 8,431,360 and U.S. Pat. No. 8,846,352.

Growth of algae and production of algal products using wood-derived lignocellulosic hydrolysates has not been disclosed. Cellulosic feedstocks for algae have been limited to non-wood sources. US Patent Application Publication No. 20110306100 discloses the use of switchgrass and corn stover following fungal enzymatic digestion for obtaining algal fatty acids. US Patent Application Publication No. 20100151538A1 discloses the use of depolymerized cellulosic material selected from the group consisting of corn stover, Miscanthus, forage sorghum, sugar beet pulp, and sugar cane bagasse for heterotrophic cultivation of Prototheca, an obligate non-pigmented microalga. US Patent Application Publication No. 20090011480 and U.S. Pat. No. 8,790,914 disclose use of depolymerized cellulosic material selected from the group consisting of corn stover, switchgrass, and sugar beet pulp for heterotrophic cultivation of microalgae. Use of rice straw, for mixotrophic cultivation of Chlorella pyrenoidosa, and of wheat bran using the microalgae Chlorella vulgaris and Scenedesmus obliquus for mixotrophic or heterotrophic cultivation have been reported. However, the methods of producing microalgal biomass and products using wood-sourced lignocellulosic hydrolysates are not disclosed. United States Patent Application Publication No. 20092117569 discloses the use of source material that originates from treated wood pulp for cultivation of yeasts. Also, the use of yeast cannot substitute for microalgae in whole composition and in terms of the production of compounds, certain compositions, yields, or mixture of compounds required for target products, such as high quality animal, insect or fish feed, nutritional proteins, polysaccharides and lipids, immunomodulatory compounds, nutritional and fiber supplements, colorants, and recombinant nucleic acids and proteins.

### BRIEF SUMMARY OF THE INVENTION

An embodiment of the invention provides a method of producing a culture medium for culturing a microbe to produce a product of interest. The method of producing a culture medium comprises the steps of:

a) providing a lignocellulosic biomass, wherein the lignocellulosic biomass comprises a lignocellulosic compound,

b) hydrolyzing the lignocellulosic biomass to produce a lignocellulosic hydrolysate, wherein the lignocellulosic hydrolysate comprises a simplified sugar produced from at least a portion of the lignocellulosic compound,

c) optionally, separating the lignocellulosic hydrolysate into a first portion and a second portion and treating the second portion of the lignocellulosic hydrolysate to convert a portion of the lignocellulosic compound and/or the simplified sugar to a non-sugar agent;

d) mixing the treated second portion of the lignocellulosic hydrolysate comprising the non-sugar agent with the first portion of the lignocellulosic hydrolysate,

e) producing a culture medium comprising the mixture obtained after step d).

In one embodiment, the non-sugar agent is an organic acid, an alcohol, a micronutrient, a salt, a saponifiable or fatty acid compound, a furfural, process water, a protein, or any combination thereof.

The non-sugar agent can be an organic acid such as acetic acid, propionic acid, citric acid, fumaric acid, glycolic acid, lactic acid, malic acid, pyruvic acid, succinic acid, glucuronic acid, galacturonic acid, and ferulic acid.

A further embodiment of the invention also provides a culture medium produced according to the method described above, which is hereinafter referred to as “a method of producing a medium containing lignocellulosic hydrolysate.”

A further embodiment of the invention provides a method for synthesizing a product of interest using fermentation. In one embodiment, the method comprises the steps of:

a) providing a culture medium produced according to the method of producing a medium containing lignocellulosic hydrolysate;

b) providing a microalgal cell that produces the product of interest;

c) culturing the microalgal cell in the culture medium to produce a microalgal culture from the microalgal cell; and

d) purifying the product of interest from the microalgal culture.

### BRIEF DESCRIPTION OF DRAWINGS

FIG. 1. shows the hemicelluloses and/or lignin origin of many process residuals. Taken from Jonsson and Martin (2016).

FIG. 2. Overall production process for producing microalgal products using wood-derived lignocellulosic hydrolysates. Key components in the process are highlighted. [10] Providing a culture medium comprising wood-derived lignocellulosic hydrolysate with a simplified sugar in the presence of a non-sugar agent; [20] choosing to convert (Y) some of that hydrolysate into a wood-derived organic acid, to produce a feedstock stream enriched for a specific non-sugar agent, which can be optionally provided [30] into the culture medium; and/or choosing to not convert (N) some of that hydrolysate into an organic acid; [40] providing a microalgal cell, and optionally a second type of microbial cell, to produce a culture by a fermentation [50]. Algal cells can be selectively grown on hydrolysate with process residuals and/or on an enriched feedstock stream of wood-derived organic acid to generate product and even alter the product of interest [60], which is then purified to produce the desired microalgae-derived target products [70].

FIG. 3. OD750 profiles of KAS908 (Chlorella sorokiniana) grown heterotrophically in three wood hydrolysates in replicated 96-well plates: Southern Hardwood Chips (SHC), Southern Pine Bleached Kraft (SPBK) and Southern Pine Finer chips (SPFC).

FIG. 4. Comparison of heterotrophic growth in replicated 50-mL flasks measured by OD750 absorbance of KAS740 (Scenedesmus armatus) on Southern Pine Finer Chips (SPFC) hydrolysate and equivalent glucose concentration.

FIG. 5. Heterotrophic growth of KAS1101 (Rhodotorula glutinis ATCC 2527) in replicated 96-well plates using different concentrations of Southern Hardwood Chip (SHC) hydrolysates with Yeast Extract-Peptone (YP) nutrients or YP medium with 20 g/L glucose.

FIG. 6. Growth and sugar utilization (glucose and xylose uptake monitored by HPLC) of KAS908 (Chlorella sorokiniana) under heterotrophic fermentation in a) BSP (Bleached Southern Pine) wood hydrolysates and b) C5 and C6 model sugars standardized to total sugars in BSP wood hydrolysates, performed in a 7-L batch fermentor.

FIG. 7. Thin layer chromatographs for phospholipids, phosphatidylcholine (PC) and phosphatidylethanolamine (PE), from KAS908 (Chlorella sorokiniana) biomass grown heterotrophically in medium with varying glucose:nitrogen (w/w) ratios at day 3 of growth: Columns 1-7 show extracts from biomass in dark shake flask using various glucose:nitrogen (w/w) ratios: 1=1:1, 2=3:1; 3=4:1; 4=5:1; 5 =7:1; 6=9:1; 7=13:1; 8=KAS908 in F/2 (photosynthetic control under lights); Columns 9-12 show extracts from biomass in dark 6-L fermentor: 9=4:1 glucose to nitrogen ratio; 10=model sugars 19.43 glucose and 8.06 g/L xylose in 2F; 11=Bleached Southern Pine lignocellulosic hydrolysate; and 12=heterotrophic 2F+36 g/L glucose.

FIG. 8. Glucose utilization of KAS908 (Chlorella sorokiniana) and KAS1101 (Rhodotorula glutinis) using Southern Hardwood Chips (SHC) lignocellulosic hydrolysate sugars.

### DETAILED DESCRIPTION OF THE INVENTION

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “about” or “approximately” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed. In the context of compositions containing amounts of ingredients where the terms “about” or “approximately” are used, these compositions contain the stated amount of the ingredient with a variation (error range) of 0-10% around the value (X±10%).

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. When ranges are used herein, combinations and subcombinations of ranges (e.g., subranges within the disclosed range), specific embodiments therein are intended to be explicitly included.

The term “photoautotrophs” refers to an organism capable of synthesizing its own food from inorganic substances using light as an energy source. Examples of photoautotrophs include green plants and photosynthetic bacteria.

The term “facultative” refers to an organism that is capable of but not restricted to a particular mode of life. For example, a facultative anaerobe can synthesize ATP by aerobic respiration if oxygen is present, but is capable of fermentation or anaerobic respiration if oxygen is absent.

The term “facultative heterotroph” refers to a photoautotrophic organism that is also capable of utilizing organic compounds for growth and/or maintenance and/or survival when light energy is not sufficient or is absent. The term also encompasses facultative heterotrophs and descendants thereof that lose their capability to perform photosynthesis, or acquire defects that result in their inability to grow as phototrophs, or are enabled to grow in the dark through genetically engineering, including for trophic conversion or for utilization of the preferred carbon feedstock. The term “obligate heterotroph” refers to a cell that is unable to perform photosynthesis and requires an exogenous feedstock for survival. Some representative facultative heterotrophs and obligate heterotrophs that can grow in the dark in the presence of a carbon source are used in the method of the invention in a non-limiting manner. Examples of other microalgae are given below.

The term “axenic” refers to the state of a culture in which only a single species, variety, or strain of an organism is present and wherein the culture is free of all other organisms.

The term “biomass” as used herein refers to a mass of living or non-living biological material and its derivatives and includes both natural and processed, as well as natural organic materials more broadly. Thus, “microalgal biomass,” and “algal biomass” refers to material produced by growth and/or propagation of microalgal cells. “Woody biomass” refers to biomass from trees and shrubs. “Lignocellulosic biomass” refers to biomass comprising lignocellulose, for example, wood.

“Biomass production” or “biomass accumulation” means an increase in the total number or weight of the cells of the organisms that are present in a culture over time. Biomass is typically comprised of cells; intracellular contents as well as extracellular material such as may be secreted or evolved by a cell; and can also be processed such that a fraction of the biomass is removed leaving residual biomass.

“Biorefinery” means a facility that integrates biomass conversion processes and equipment to produce fuels, power, and chemicals from biomass. A pulp and paper mill biorefinery uses woody biomass.

“Fed-batch fermentation” refers to a fermentation where one or more nutrients are supplied to the bioreactor during cultivation and in which the product remains in the bioreactor until the end of the fermentation run.

A “product of interest” is a substance synthesized by a cell. Examples of a product of interest include but are not limited to, proteins, lipids, carbohydrates, biogases, volatile materials, sugars, amino acids, isoprenoids, terpenes, or precursor thereof. Such substances may be synthesized constitutively by the organisms throughout growth and the amount of the substance in the culture may increase simply due to an increase in the number of organisms. Alternatively, the synthesis of such substances may be induced or altered in response to culture conditions or other environmental factors, for example, nitrogen starvation or elevated ammonium levels, or components from cellulosic hydrolysates.

“Protein” refers to full-length protein polymers or peptide fragments thereof. As non-limiting examples, protein as peptides can be antibiotics or promoters of gene expression. Protein can be used in whole biomass or delipidated microalgal meal for animal and fish feed.

The product of interest can also be an amino acid. An amino acid can have nutritional value, for example, taurine.

The product of interest can also be a polysaccharide. A polysaccharide can have health value, for example as immunomodulatory, macrophage-stimulating or humectant properties such as beta-glucan or undefined exopolysaccharides.

The amount of a product of interest accumulated over time relative to the culture volume and relative to their original amount is considered as “product accumulation” that can be measured or quantified such as by specific productivity or on a relative basis compared to a control culture.

The term “conditions favorable to cell division” or “conditions favorable to vegetative growth” mean conditions in which cells divide at a pace such that an industrial production run is completed in about 60 to 168 to 240 hours, preferentially in less than 240, 144, 120 or 96 hours, including a lag time of less than about 24 hours.

The term “co-culture”, and variants thereof such as “co-cultivate,” refer to the presence of two or more types of cells in the same fermentor or bioreactor. The two or more types of cells may both be microorganisms, such as microalgae, or may be a microalgal cell cultured with a different cell type. The culture conditions may be those that promote growth and/or propagation of the two or more cell types or those that facilitate growth and/or proliferation of one, or a subset, of the two or more cells types while maintaining cellular growth for the remainder.

The term “cultivated” or “cultivation” or “culturing” refers to the purposeful fostering of growth (increases in cell size, cellular contents, and/or cellular activity) and/or propagation (increases in cell numbers via mitosis) of one or more microbial or microalgal cells by use of intended culture conditions. The combination of both growth and propagation may be termed proliferation. Examples of intended conditions include the use of a defined medium (with known characteristics such as pH, ionic strength, and carbon source), specified temperature, oxygen tension, and growth in a fermentor or bioreactor. The term does not refer to the growth of microorganisms in nature or otherwise without intentional introduction or human intervention, such as natural growth of an organism.

The term “fermentor” or “bioreactor” or “fermentation vessel” or “fermentation tank” means an enclosed vessel or partially enclosed vessel in which cells are cultivated or cultured, optionally in liquid suspension. A fermentor or bioreactor of the disclosure includes non-limiting embodiments such as an enclosure or partial enclosure that permits cultured cells to be exposed to light or which allows the cells to be cultured without the exposure to light. The term “port”, in the context of a vessel that is a fermentor or bioreactor, refers to an opening in the vessel that allows influx or efflux of materials such as gases, liquids, and cells. Ports are usually connected to tubing leading from the fermentor or bioreactor.

The term “fermenter” refers to an organism that causes fermentation.

The term “fixed carbon source” means a compound containing carbon that can be used as a source of carbon and/or energy by an organism. Typically, a fixed carbon source exists at ambient temperature and pressure in solid or liquid form.

The term “organic acid” refers to one or more molecules that are organic compounds with acidic properties. The most common organic acids are the carboxylic acids. A “carboxylic acid” contains a carboxyl group distinct from sugar carbohydrates such as glucose commonly used in algal fermentation. Acetic acid is a two-carbon carboxylic acid, CH₃COOH, commonly used in chemical manufacturing. As a chemical reagent, acetic acid is manufactured from petrochemical feedstock. Propionic acid (propanoic acid) is a carboxylic acid with the chemical formula CH₃CH₂COOH. The anion CH₃CH₂COO— as well as the salts and esters of propionic acid are known as propionates (or propanoates). Other such acids can include but are not limited to citric, fumaric, glycolic, lactic, malic, pyruvic, and succinic acids.

“Sugar acids” and “chlorogenic acids” are also organic acids and can include but are not limited to glucuronic, galacturonic and other uronic acids, and ferulic, with a carboxylic acid functional group such as obtained in lignocellulosic derivatives. Organic acids can be used alone or in combination, such as in combinations that may occur naturally in lignocellulosic derivatives.

Bio-based organic acids can be sourced from microbial anaerobic or partial anaerobic digestion or fermentation processes as is known in the art.

The terms “heterotrophic conditions” and “heterotrophic fermentation” and “dark heterotrophic cultivation” or “dark heterotrophic culture” refer to the presence of at least one fixed carbon source and the absence of light during fermentation. “Mixotrophic fermentation” refers to cultivation in the presence of at least one fixed carbon source and the presence of light during fermentation.

“Lignocellulosic hydrolysis” or “saccharification” refers to a process of converting cellulosic or lignocellulosic biomass into monomeric sugars or monosaccharides, such as the hexose, glucose, and the pentose, xylose. “Saccharified” or “simplified” or “depolymerized” cellulosic or lignocellulosic material or biomass refers to cellulosic or lignocellulosic material or biomass that has been converted into monomeric sugars through saccharification. Saccharification also produces oligosaccharides that are oligomeric, short-chain polymers of monomeric sugars. Some sugars are C12 dimers composed of two C6 sugars. These dimers can also be a starting point for an engineered or for a natural algae or other microbe and/or for an algal/microbial combination.

Solid state fermentation of woody biomass by fungi or polycultures is one process known in the art to produce hydrolytic enzymes which subsequently produce sugar-rich and even nitrogen-rich streams, either in phased steps or in simultaneous saccharification as feedstock for algal heterotrophic or mixotrophic culture.

“Model sugar” or “purified sugar” refers to monomeric or oligomeric sugars that are individual sugars, separate from other sugars, in a pure or reagent grade compound.

“Lignocellulosic hydrolysate” or “cellulosic hydrolysate” refers to the products of saccharification and the process residuals.

“Process residuals” or “process impurities” and “process inhibitors refers to non-monosaccharide and non-oligosaccharide residuals from the wood lignocellulosic hydrolysis process, comprising but not limited to compounds selected from organic acids (e.g., acetic, formic, levulinic), aldehydes (e.g., furfural, 5-hydroxymethylfurfural, vanillin), lignins, lignin byproducts or derivatives, inorganic salts (e.g., sulfates, phosphates, hydroxides), alcohols, fatty acids, fatty alcohols, fats, waxes, polyesters (e.g., suberin), terpenoids, alkanes, wood extractives, Hibbert's ketones, and proteins; where the organic acids may further comprise citric, fumaric, glycolic, lactic, malic, proprionic, pyruvic, succinic, glucuronic, galacturonic, uronic, chlorogenic, or lignocellulosic acid; and where the solvent water is also considered a process residual.

The term “feedstock” refers to nutritional material assimilated or metabolized by a cell.

The term “isoprenoid” or “terpenoid” or “terpene” or “derivatives of isoprenoids” refers to any molecule derived from the isoprenoid pathway with any number of 5-carbon isoprene units, including compounds that are monoterpenoids and their derivatives, such as carotenoids and xanthophylls. The isoprenoid pathway generates numerous commercially useful target compounds, with non-limiting examples such as pigments, terpenes, vitamins, fragrances, flavorings, solvents, steroids and hormones, lubricant additives, and insecticides. These in turn are used in products for food and beverages, perfumes, feed, cosmetics, and raw materials for chemicals, nutraceuticals, and pharmaceuticals.

The term “carotenoid” refers to a compound composed of a polyene backbone which condensed from five-carbon isoprene unit, “carotenoid” can be an acyclic, or one (monocyclic) or two and it can be terminated by cyclic end-groups of the number (bicyclic). The term “carotenoid” may include both carotenes and xanthophylls.

A “carotene” refers to a hydrocarbon carotenoid. “Xanthophylls” are oxygenated carotenoids. Modification of pyrophosphate and phosphate groups of isoprene derivatives include oxidations or cyclizations to yield acyclic, monocyclic and bicyclic terpenes including monoterpenes, diterpenes, tripterpenes, or sequiterpenes, etc.

“Lipids” refers to any of a large group of organic compounds that are oily to the touch and insoluble in water. Lipids include fatty acids, oils, waxes, sterols, polar lipids, neutral lipids, phospholipids, and triglycerides. They are a source of stored energy and are a component of cell membranes. Phospholipids are a lipid containing a phosphate group in its molecule. They include diacylglyceride structures, e.g., phosphatidic acid (phosphatidate; PA), phosphatidylethanolamine (cephalin; PE), phosphatidylcholine (lecithin; PC), phosphatidylserine (PS), phosphosphingolipids, and glycerophospholipids. “PUFA” or “PUFAs” refers to lipids that are polyunsaturated fatty acids. Examples of PUFAs are docosahexaenoic acid (DHA, represented as 22:6 n-3); eicosapentaenoic acid (EPA, represented as 20:5 n-3); omega-3 docosapentaenoic acid (DPA n-3, represented as 22:5 n-3); omega-6 arachidonic acid (ARA, represented as 20:4 n- 6); and omega-6 docosapentaenoic acid (DPA n-6, represented as 22:5 n-6).

The term “microorganism” or “microbe” refers to microscopic unicellular organisms, including microalgae, which can also be filamentous or colonial. The microorganisms usable in the fermentation according to the present invention can include mutants, naturally occurring strains selected for a specific characteristic, or genetically engineered variants of a naturally occurring strain.

The term “microalgae” refers to a eukaryotic microorganism that contains a chloroplast, and optionally is photosynthetic, or a prokaryotic microorganism capable of being photosynthetic. Microalgae include obligate photoautotrophs, which are incapable of metabolizing a fixed carbon source as energy, as well as obligate or facultative heterotrophs, which are capable of metabolizing a fixed carbon source. Microalgae as obligate heterotrophic microorganisms include those that have lost the ability of being photosynthetic and may or may not possess a chloroplast or chloroplast remnant. Microalgae can divide to produce populations of cells and can be scaled-up or enter a production phase to produce biomass, and this process can be continued indefinitely until a maximum productivity is achieved.

The term “recombinant” when used with reference, a cell, nucleic acid, protein, or vector, indicates that the cell, nucleic acid, protein or vector, has been modified from its natural state. For example, a recombinant cell comprises an exogenous nucleic acid or protein or the alteration of a native nucleic acid or protein, or is derived from a cell or organism or micro-organism so modified.

The term “robust” or “robust culture”, in the context of selected strains or lines of a species, refer to a population of algae that contain a desired phenotype and equal or greater growth characteristics, especially under heterotrophy, compared to the original strain.

A method for use of wood-derived lignocellulosic hydrolysate directed towards accumulation of sufficient biomass, target compound, or improved compound profile by a microalgae species will have economic benefits and for the first time, demonstrate using a non-seasonal agricultural resource that is available all year round for efficient operations at a mill-based biorefinery. In particular, methods to produce and modify levels of target compounds are desirable for optimizing efficient industrial heterotrophic fermentation that is independent of weather, climates, seasons, and geography. Target compounds of value include proteins, lipids, carotenoids/isoprenoids and recombinant molecules. The latter may be compounds which favor rapid biomass growth for their expression and accumulation. Surprisingly, it was discovered that the mixture of sugar and non-sugar agents in the wood lignocellulose hydrolysate favors production of algal product biomass over use of pure sugars alone; shortens fermentation cycle time, increases yield; alters protein yield and composition; alters lipid yield and composition; supports recombinant gene expression; induces and supports certain pigment accumulation in a dark fermentation; reduces certain other pigment accumulation in a dark fermentation; and enables co-culture of two different species to fully utilize the fixed carbon component.

In one embodiment, the invention provides that not only does wood-derived lignocellulosic hydrolysate support algal growth, but some algal species also perform better in the presence of unpurified wood hydrolysate and the resulting products can differ in several ways. For example, wood lignocellulosic sugars are shown to be completely utilized (fully depleted) in the culture solutions. Also, the efficiency of conversion of hydrolysate into biomass is measured to demonstrate the impact of process residuals, in addition to the sugars, for producing algal biomass and product. This is an essential feature that must be monitored to decide if a specific microbial bioconversion method warrants implementation at a mill biorefinery site.

Accordingly, for the purposes of this invention, “a wood processing operation” refers to an industry that processes wood for various uses, for example, printing and writing paper grades, various coated and uncoated specialties and paper grades, tissue and toweling products, paperboard, medical packaging, absorbent and air laid non-woven products (such as diapers, hygiene, incontinence products), textile fibers, film, and sawn timber. These products utilize many types of woody biomass that may comprise but are not limited to Northern Softwood (for example Lodgepole Pine, White/Engelmann Spruce, Jack Pine, Sitka Spruce, Norway Spruce, and Black Spruce); Northern Hardwood (for example Maple, Birch, Poplar); Southern Softwood (for example Loblolly Pine, Shortleaf Pine); Southern Hardwood (for example Oak, Maple and Poplar). The other woody biomass in the supply chain comprises but is not limited to debarking residues, chip screening residues, knots and pulp fibers. The lignocellulosic biomass can also be a byproduct from the wood-processing operation. The associated mills can be of various types and can include chemical pulp mills (such as sulfate mills and sulfite mills) and chemical-mechanical pulp mills (such as TMP and CTMP mills). Additional embodiments of wood processing operations that supply biomass containing lignocellulose can be identified and used according to the methods described herein by a person of ordinary skill in the art and such embodiments are within the purview of the invention.

Thus, the invention provides improved methods for producing algal product from woody feedstocks, particularly for methods that provide a means to produce target products, and preferred profiles, using obligate and facultative heterotrophs, with and without pigments, with greater yield and efficiency. The present invention meets this need for this non-commonly used wood-derived lignocellulosic hydrolysate feedstock with exemplification for several algal products.

An embodiment of the invention provides processing a lignocellulosic biomass, wherein the lignocellulosic biomass is raw material for a wood processing operation. The lignocellulosic biomass can be wood or byproduct from the wood-processing operation.

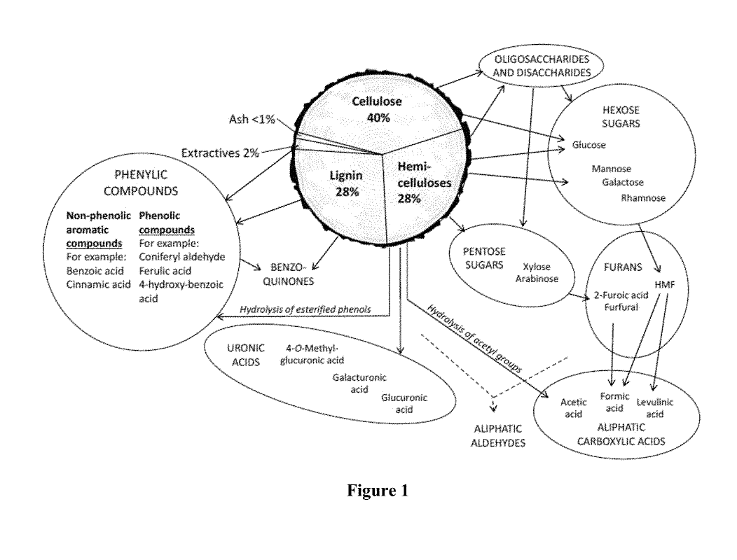

The major chemical constituents of softwoods and hardwoods used in a wood processing operation are shown in Table 1 and FIG. 1, with hardwood have much higher hemicelluloses and much lower lignin than softwood.

Softwood and hardwood as used herein refer to the physical structure and makeup of the wood, i.e., hardwoods is hard and durable; whereas, compared to hardwood, softwood is soft and workable. Hardwood typically comes from angiosperm—or flowering plants—such as oak, maple, or walnut, that are not monocots. Softwood typically comes from gymnosperm trees, usually evergreen conifers, like pine or spruce.

Lignocellulose-derived process residuals from a typical softwood, Norway Spruce used in a wood processing operations is also shown in Table 1 and FIG. 1. However across the various wood species there can be a range of chemical composition values for both wood and for bark as shown in Table 2. Various chemical compositions are shown in Table 3 for some North American woods. Specific chemical composition within a species for wood, bark and knotwood for Scots Pine, a softwood, is shown in Table 4.

Species variations among softwood lignins are relatively negligible in contrast with hardwood lignins. For the hemicelluloses, softwood and hardwoods are quite different chemically, as is known in the art. The different hemicellulosic polysaccharides for the two groups show various hydrolysis rates to produce different yield amounts of degradation sugars using the same process conditions. Norway Spruce typifies the generalized softwood profile. Upon degradation of Norway Spruce by pretreatment, numerous categories of constituents can result; most of the lignocellulose-derived inhibitors in the process residuals form when hemicelluloses and/or lignin are solubilized and degraded (Table 1 and FIG. 1).

In addition, new compounds are still being identified as inhibitors in lignocellulosic biomass hydrolysates. Some compounds function as co-inhibitors, producing negative synergistic effects (with no negative effect as individual compounds) that affect longer lag phase, slower growth, lower cell density, and lower product yield with lower glucose consumption (Zha et al. 2014).

TABLE 1
A generalized chemical composition of softwoods and hardwoods.

| | Cellulose | Hemicellulose | Lignin |
|---|---|---|---|
| Softwoods* | 43% | 28% | 29% |
| Hardwoods | 45% | 34% | 21% |

*Norway Spruce typifies the generalized softwood profile.

TABLE 2
Ranges for constituents by mass of lignin, polysaccharide, extractive
and ash in woods and barks. See, world-wide website:
carbolea.ul.ie/wood.php and USDA (1971).

| | Softwoods | | Hardwoods | |
|---|---|---|---|---|
| Component | Wood | Bark | Wood | Bark |
| Lignins | 25-30 | 40-55 | 18-25 | 40-50 |
| Polysaccharides | 66-72 | 30-48 | 74-80 | 32-45 |
| Extractives | 2-9 | 2-25 | 2-5 | 5-10 |
| Ash | 0.2-0.6 | Up to 20 | 0.2-0.6 | Up to 20 |

Bark as a woody biomass is quite heterogeneous and chemically complex. Compared to wood, bark has elevated levels of ash, lignin, and extractives and lower levels of polysaccharides. Extractives in bark are both much more abundant, more variable, and also unique than they are in wood. Bark extractives comprise lipophilic fractions (e.g., fats, waxes, terpenes and terpenoids, and higher aliphatic alcohols) and the more abundant hydrophilic fractions (e.g., phenolic constituents). Oligosaccharides include about 60-70% glucose, 5-15% xylose, 5-10% arabinose, and 3-4% each of galactose and mannose, with raffinose and stachyose present in minor amounts in bark (USDA 1971). Chemical composition of knots and different fractions of wood is shown for Scots Pine in Table 4. The highest lignin content and extractives content (9%) was determined for knotwood (32%).

Nevertheless, bark, knotwood, and the other wood fractions from Scots pine that differed significantly in chemical composition, were shown to be relatively similar with regard to susceptibility to pretreatment and enzymatic saccharification. The most obvious difference with regard to enzymatic saccharification was that without pretreatment the bark fraction was more susceptible than the other fractions. This similarity in saccharification outcome is considered favorable for the utilization of wood operation residues from Scots pine for bioconversion to fuels and to chemicals.

TABLE 3
Chemical composition of some North American woods. See, world-wide website:
web.nchu.edu.tw/pweb/users/taiwanfir/lesson/9324.pdf

| | Percentage | | | | | | | | |
|---|---|---|---|---|---|---|---|---|---|
| Species | Glucose | Xylose | Galactose | Arabinose | Ma [symbol] | U [symbol] | Acetyl | Lignin | Ash |
| Red Maple | 46 | 19 | 0.6 | 0.5 | 2.4 | 3 [symbol] 5 | 3.8 | 24 | 0.2 |
| Sugar Maple | 52 | 15 | <0.1 | 0.8 | 2.3 | 4.4 | 2.9 | 23 | 0.3 |
| Yellow Birch | 47 | 20 | 0.9 | 0.6 | 3.6 | 4.2 | 3.3 | 21 | 0.3 |
| Paper Birch | 4 [symbol] | 26 | 0.6 | 0.5 | 1 [symbol] | 4 [symbol] 6 | 4.4 | 19 | 0.2 |
| Beech | 46 | 19 | 1.2 | 0.5 | 2.1 | 4.8 | 3.9 | 22 | 0.4 |
| Sweetgum | 39 | 1 [symbol] | 0.8 | 0.3 | 3.1 | — | — | 24 | 0.2 |
| Quaking Aspen | 49 | 17 | 2.0 | 0.5 | 2.1 | 4.3 | 3. [symbol] | 21 | 0.4 |
| Southern Red Oak | 41 | 19 | 1.2 | 0.4 | 2.0 | 4.5 | 3. [symbol] | 24 | 0. [symbol] |
| American Elm | 52 | 12 | 0.9 | 0.6 | 2.4 | 3.6 | 1. [symbol] | 24 | 0. [symbol] |
| Balsam Fir | 46 | 6.4 | 1.0 | 0 [symbol] 5 | 12 | 3.4 | 1.3 | 29 | 0.2 |
| White Spruce | 45 | 9.1 | 1.2 | 1.5 | 11 | 3.6 | 1.3 | 27 | 0.3 |
| Black Spruce | 44 | 6.0 | 2.0 | 1.5 | 9.4 | 5.1 | 1.2 | 30 | 0.3 |
| Jack Pine | 46 | 7.1 | 1.4 | 1 [symbol] 4 | 10 | 3.9 | 1.2 | 2 [symbol] | 0.2 |
| Red Pine | 42 | 9.3 | 1.8 | 2.4 | 7.4 | [symbol] .0 | 1.2 | 29 | 0.4 |
| Eastern White Pine | 45 | 6.0 | 1.4 | 2.0 | 11 | 4.0 | 1.2 | 29 | 0.2 |
| Loblolly Pine | 45 | 6.8 | 2.3 | 1.7 | 11 | 3. [symbol] | 1.1 | 28 | 0.3 |
| Northern White Cedar | 43 | 10.0 | 1.4 | 1.2 | 8.0 | 4.2 | 1.1 | 31 | 0.2 |
| Eastern Hemlock | 44 | [symbol] | 1.2 | 0.6 | 11 | 3.3 | 1. [symbol] | 33 | 0.2 |

^{a}Based on extractive-free wood. [symbol]
indicates data missing or illegible when filed

TABLE 4
Chemical composition of fractions of Scots pine^{a}.

| | Contents in % (w/w) | | | | | | | | | | |
|---|---|---|---|---|---|---|---|---|---|---|---|
| | Arabinan ± | Galactan ± | Glucan ± | Mannan ± | Xylan ± | Klason lignin ± | Acid soluble | Total lignin ± | Extractives ± | Ash ± | |
| Fraction | SD | SD | SD | SD | SD | SD | lignin ± SD | SD | SD | SD | Total^{b} |
| Juvenile | 2.0 ± 0.1 | 3.1 ± 0.1 | 42.7 ± | 11.8 ± 0.2 | 6.6 ± | 27.9 ± 0.3 | 1.6 ± 0.1 | 29.5 ± 0.4 | 4.5 ± 0.5 | 0.2 ± | 100 |
| heartwood | | | [symbol] .9 | | 0.1 | | | | | 0.1 | |
| Mature | 1.8 ± 0.1 | 3.0 ± 0.1 | 42.2 ± | 12.1 ± 0.2 | 5.3 ± | 27.7 ± 0.1 | 1.4 ± 0.1 | 29.1 ± 0.2 | 4.4 ± 0.6 | 0.2 ± | 98 |
| heartwood | | | 5.2 | | 0.1 | | | | | 0.1 | |
| Juvenile | 1.9 ± 0.1 | 2.8 ± 0.2 | 39.7 ± | 10.5 ± 0.3 | 6.2 ± | 25.8 ± 0.4 | 1.7 ± 0.1 | 27.4 ± 0.5 | 3.0 ± 0.2 | 0.3 ± | 92 |
| sapwood | | | 0.1 | | 0.3 | | | | | 0.1 | |
| Mature | 1.7 ± 0.1 | 2.3 ± 0.1 | 41.8 ± | 14.0 ± 0.2 | 5.5 ± | 26.9 ± 0.2 | 1.5 ± 0.1 | 28.4 ± 0.2 | 2.9 ± 0.1 | 0.2 ± | 97 |
| sapwood | | | 1.6 | | 0.1 | | | | | 0.1 | |
| Bark | 2.4 ± 0.2 | 2.8 ± 0.2 | 41.8 ± | 11.7 ± 0.3 | 5.4 ± | 28.2 ± 0.3 | 1.9 ± 0.1 | 30.0 ± 0.3 | 3.5 ± 0.2 | 0.9 ± | 99 |
| | | | 2.0 | | 0.1 | | | | | 0.3 | |
| Top parts | 2.0 ± 0.1 | 3.4 ± 0.1 | 41.4 ± | 11.4 ± 0.1 | 6.6 ± | 28.1 ± 0.2 | 1.6 ± 0.1 | 29.7 ± 0.2 | 3.3 ± 0.6 | 0.3 ± | 98 |
| | | | 1.4 | | 0.1 | | | | | 0.1 | |
| Knorwood | 2.2 ± 0.1 | 4.1 ± 0.3 | 38.2 ± | 11.9 ± 0.1 | 6.6 ± | 30.0 ± 0.3 | 1.6 ± 0.1 | 31.5 ± 0.3 | 9.2 ± 0.3 | 0.3 ± | 105 |
| | | | 1.4 | | 0.1 | | | | | 0.1 | |

^{a}Yield in g 100 g⁻¹ wood/bark. Mean values and standard deviations based on three replicates.
^{b}Includes arabinan, galactan, glucan, mannan, xylan, total lignin, extractives, and ash. [symbol]
indicates data missing or illegible when filed

An embodiment of the invention provides a method of treating lignocellulosic biomass, for example, a biomass that is raw material for a wood processing operation. The method of treating a lignocellulosic biomass comprises the steps of:

a) providing a lignocellulosic biomass, wherein biomass comprises a lignocellulosic compound,

b) hydrolyzing the lignocellulosic biomass to produce a lignocellulosic hydrolysate, wherein the lignocellulosic hydrolysate comprises a simplified sugar produced from at least a portion of the lignocellulosic compound,

c) optionally, separating the lignocellulosic hydrolysate into a first portion and a second portion and treating the second portion of the lignocellulosic hydrolysate to convert a portion of the lignocellulosic compound and/or the simplified sugar to a non-sugar agent;

d) mixing the treated second portion of the lignocellulosic hydrolysate comprising the non-sugar agent with the first portion of the lignocellulosic hydrolysate.

A further embodiment of the invention also provides a method of producing a culture medium for culturing a microbe to produce a product of interest. The method of producing a culture medium comprises the steps of:

a) providing a lignocellulosic biomass, wherein the lignocellulosic biomass comprises a lignocellulosic compound,

b) hydrolyzing the lignocellulosic biomass to produce a lignocellulosic hydrolysate, wherein the lignocellulosic hydrolysate comprises a simplified sugar produced from at least a portion of the lignocellulosic compound,

c) optionally, separating the lignocellulosic hydrolysate into a first portion and a second portion and treating the second portion of the lignocellulosic hydrolysate to convert a portion of the lignocellulosic compound and/or the simplified sugar to a non-sugar agent;

d) mixing the treated second portion of the lignocellulosic hydrolysate comprising the non-sugar agent with the first portion of the lignocellulosic hydrolysate,

e) producing a culture medium comprising the mixture obtained after step d).

An embodiment of the invention also provides a culture medium produced according to the method described above, which, as noted above, is referred to as “a method of producing a lignocellulosic hydrolysate containing medium.”

In an embodiment, the step of hydrolysis is performed using a hydrolytic enzyme, preferably an enzyme that hydrolyses lignin, lignocellulose, or cellulose, for example, ligninase, lignocellulase, hemicellulose, or cellulase. Conditions appropriate for an enzyme used for the hydrolysis of lignin, lignocellulose, and cellulose are well known in the art and can be appropriate used by a skilled artisan.

The term “hydrolytic enzyme(s)” is meant to refer to enzymes that catalyze hydrolysis of biological materials such as cellulose. Hydrolytic enzymes include “cellulases,” which catalyze the hydrolysis of cellulose to products such as glucose, cellobiose, cello-oligodextrins, and other cello-oligosaccharides. “Cellulase” is meant to be a generic term denoting a multienzyme complex or family, including exo-cellobiohydrolases (CBH), endoglucanases (EG), and β-glucosidases (βG) that can be produced by a number of plants and microorganisms. Many crude cellulase extracts also include some hemicellulases. The process in accordance with embodiments of the invention may be carried out with any type of cellulase enzyme complex, regardless of their source; however, microbial cellulases are generally available at lower cost than those of plants. Among the most widely studied, characterized, and commercially produced cellulases are, e.g., those obtained from fungi of the genera Aspergillus, Humicola, and Trichoderma, and from the bacteria of the genera Bacillus and Thermobifida. Also, for example, cellulase produced by the filamentous fungi Trichoderma longibrachiatum includes at least two cellobiohydrolase enzymes termed CBHI and CBHII and at least 4 EG enzymes.

The non-sugar agent can be an organic acid such as acetic acid, propionic acid, citric acid, fumaric acid, glycolic acid, lactic acid, malic acid, pyruvic acid, succinic acid, glucuronic acid, galacturonic acid, and ferulic acid.

In some embodiments, the culture medium contains process residuals from the wood lignocellulosic hydrolysis process that comprise non-sugar agents. Use of process residuals from the wood lignocellulosic hydrolysis process that comprise non-sugar agents according to the invention provides enhanced growth of the algal biomass relative to an algal culture control grown in a medium that lacks the non-sugar agents.

An embodiment of the invention also provides a culture medium which contains process residuals from the wood lignocellulosic hydrolysis process that comprise a non-sugar agent. In one embodiment, the non-sugar agent is an organic acid, an alcohol, a micronutrient, a salt, a saponifiable or fatty acid compound, a furfural, process water, a protein, or any combination thereof. In a further embodiment, the organic acid is acetic acid. The acetic acid present in the culture medium can be produced by the lignocellulosic hydrolysis or by a microbial conversion solution wherein the microbe has produced the acetic acid from the lignocellulosic hydrolysate. In one embodiment, the acetic acid is present with at least one other fixed carbon source, for example, a sugar. In certain embodiments, the organic acid is propionic acid, citric acid, fumaric acid, glycolic acid, lactic acid, malic acid, pyruvic acid, succinic acid, glucuronic acid, galacturonic acid, or ferulic acid.

The product of interest can be biomass or a product present in biomass. Microalgae are a valuable biocatalyst for the conversion of hydrolysates into compounds of preferred compositions, including for biomass, lipids, proteins, pigments, and biomass containing recombinant genes. Algal biomass and extracts from several different species are edible and used in nutritional supplementation or coloration with affirmed GRAS status in the US. Other biomass contains protein comprised of all the essential amino acids and useful for animal feed including aquatic species feed. Yet other biomass is oil-rich and useful for bioenergy or for fractionation for obtaining polyunsaturated fatty acids (PUFAs), notably nutritional fatty acids or those of value for chemical modification for industrial purposes.

Lipids are a group of naturally occurring molecules that include fats, oils, vitamins (e.g., A, E, D, and K), triglycerides, diglycerides, monoglycerides, sterols, waxes and phospholipids. They have broad functionality. For example, polar lipids, notably phospholipids, can form the structural components of cell membranes. These are effective emulsifiers and emollients and thus useful in skin-penetrating carriers, food, and beverage preparations. Other lipids such as neutral lipids store energy within cells, with industrial applications for biofuels and chemical raw materials. Some lipids, called omega-3, omega-6, and omega-9 polyunsaturated fatty acids, are well known for application in animal and fish feed, food, nutritional supplements, and pharmaceutical products. This includes but is not limited to docosahexaenoic acid (DHA), eicosapentaenoic acid (EPA); omega-3 docosapentaenoic acid (DPA n-3); omega-6 arachidonic acid (ARA); and omega-6 docosapentaenoic acid (DPA n-6). Omega-3, 6-, and 9-fatty acids can be medium to long chain carbon molecules for a variety of industrial and real-world applications.

Another product of interest is microalgal biomass with biological pigments. Numerous naturally pigmented compounds, called carotenoids and xanthophylls, are used as antioxidants, anti-inflammatories, antiapogenics, feed and food colorants, or for extraction into nutritional supplements. These include β-Carotene, lutein, lycopene, astaxanthin, and fucoxanthin. Several carotogenic microalgae have been shown to be facultative heterotrophs for cultivation in the dark whereby carbon dioxide used during photosynthesis as the carbon growth source is substituted by some other carbon source dissolved in the nutrient medium. These may include Scenedesmus almeriensis, Chlorella zofingiensis, Muriellopsis sp., Chlorella sorokiniana and Chlorella protothecoides as sources of lutein. US Patent Application Publication No. 20050214897 discloses a method for production of astaxanthin from Chlorella zofingiensis in dark heterotrophic cultures. U.S. Provisional Application Ser. No. 62/356,896 discloses heterotrophic biomass production of astaxanthin-forming species of the Chlamydomonodales family.

Other products of interest are exemplified elsewhere herein.

PCT Publication WO2009035551 and US Patent Application Publication 2009064567 and the references cited therein, all of which are incorporated herein by reference in their entirety, provide organisms and some culture conditions of microbes rich in these long-chain fatty acids or other fatty acids. These microbes can be used in the methods described herein.

Heretofore, the provision for rapid heterotrophic or mixotrophic cell growth in wood lignocellulosic simplified sugar with product formation suited for commercial applications has not been successful for any facultative or obligate heterotrophic microalgae. An embodiment of the invention provides a method that in effect enables manufacturing biomass from a cell of class Chlorophyceae, Bacillariophyceae, Trebouxiophyceae, Euglenophyceae, Peridinea, Dinophyceae or Labyrinthulomycetes, or a product of interest produced by a cell of any of those classes.

For example, a further embodiment of the invention provides a method for synthesizing a product of interest using fermentation. In one embodiment, the method comprises the steps of:

a) providing a culture medium produced according to the method of producing a medium containing lignocellulosic hydrolysate;

b) providing a microalgal cell that produces the product of interest;

c) culturing the microalgal cell in the culture medium to produce a microalgal culture from the microalgal cell; and

d) purifying the product of interest from the microalgal culture.

In some embodiments, the cell is capable of depleting the sugar in the culture medium. In some embodiments, a monoculture of the microalgal cell is capable of utilizing both C5 and C6 sugars.

Certain embodiments of the invention enable co-culture with different cell types, which can include different microalgal species that do not require organic acids for heterotrophy but can preferentially utilize, and thus mitigate, accumulation of high levels of ammonium or other metabolites for rapid vegetative growth. In some embodiments, the co-culture is capable of depleting the sugar in the culture medium. In certain embodiments, the co-culture is capable of utilizing both C5 and C6 sugars.

The new method additionally enables co-culture with different cell types that can be a microalgal species and a yeast species for complete utilization of pentose and hexose sugars for various wood-derived feedstocks. In some embodiments, the co-culture is capable of utilizing both C5 and C6 sugars.

The heterotrophic microalgal product, either extracted or the biomass, can be used for animal feed, human nutrition and nutritional supplements, personal care, colorant, bioenergy, or recombinant gene targets. By these means, the myriad of critical advantages gained by large-scale fermentative algal culture can be realized for this vast potential supply of wood-derived carbon feedstock for production of proteins, lipids, carotenoids, recombinant gene target, and other products.

The use of indoor fermentation vessels for heterotrophy as described herein offers a new solution for biosecurity for strains that are cultured phototrophically and outdoors at large volume. Use of fermentation tanks, especially tanks located indoors, can simplify regulatory approval of the industrial scale manufacture of recombinant products.

In one embodiment of the invention, a microalgal cell is used to produce a culture. Nonlimiting examples of microalgae that can be used in accordance with the present invention include the following: Achnanthes orientalis, Agmenellum, Amphiprora hyaline, Amphora coffeiformis, Amphora delicatissima, Amphora delicatissima, Amphora sp., Anahaena, Ankistrodesmus, Ankistrodesmus falcatus, Asteracys spp., Auxenochlorella protothecoides, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri subsalsum, Chaetoceros sp., Chlamydomonas reinhardtii, Chlamydomonas dysosmos, Chlorella anitrata, Chlorella antarctica, Chlorella aureoviridis, Chlorella candida, Chlorella capsulate, Chlorella desiccate, Chlorella elupsoidea Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora, Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena, Franceia sp., Fragilaria crotonensis, Fragilaria sp., Gicocapsa sp., Gloeothamnion sp., Haematococcus spp, Heterochlorella sp., Heterosigma akashiwo, Humidophila, Hymenomonas sp., Isochrysis aff. galbana, Isochrysis galbana, Lepocinclis, Marinichlorella kessleri, Mavamaea sp., Mayamaea permitis var. pacifica, Micractinium sp., Monoraphidium minutum, Monoraphidium sp., Nannochloris sp., Nannochloropsis oceania, Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitzschia communis, Nitzschia alexandrina, Nitzschia communis, Nitzschia dissipata, Nitzschia frustulum, Nitzschia hantzschiana, Nitzschia inconspicua, Nitzscia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Ostreococcus sp., Parachlorella beijerinckii, Parachlorella kessleri, Parachlorella sp., Pascheria acidophilia, Pavlova sp., Phagus, Phormidium, Pinguiococcus pyrenoidosus Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Scenedesmus obliquus, Schizochytrium sp., Sellaphora sp., Spirogyra sp., Spirulina platensis, Stichococcus sp., Synechococcus sp., T-Isochrysis, Tetraedron sp., Tetraselmis sp., Tetraselmis suecica, Thalassiosira weissflogii, Thraustochytrium sp., Ulkenia sp., and Viridiella fridericiana.

In certain embodiments, the methods provided herein can be used for the expression of a recombinant protein or recombinant RNA by culturing a microalgal cell expressing the recombinant protein or recombinant RNA. The microalgal cell can belong to: Haematoccocus sp., for example, H. pluvialis; Chlamydomonas sp., for example, Chlamydomonas reinhardtii; Scenedesmus sp., for example Scenedesmus obliquus.

Use of co-cultures in one method of the invention could be beneficial. The cells for a co-culture can be selected such that they require an alternative carbon or nitrogen source that can be withheld or supplied when needed to modify population dynamics affecting target product production in the co-culture. Accordingly, one embodiment of the invention provides a method of using lignocellulosic feedstock for co-cultivating two cultures that are both facultative heterotrophs belonging to the class Chlorophyceae or Trebouxiophyceae. Another embodiment provides a method for co-cultivating two cultures with one being a facultative heterotroph belonging to the class Trebouxiophyceae and the other being an obligate heterotroph yeast, Rhodotorula.

In some embodiments, the culture medium contains process residuals from the wood lignocellulosic hydrolysis process that comprise non-sugar agents. Use of process residuals from the wood lignocellulosic hydrolysis process that comprise non-sugar agents according to the invention provides enhanced growth of the algal biomass relative to an algal culture control grown in a medium that lacks the non-sugar agents. In certain embodiments, culture media for members of the genera Chlorella, Scenedesmus, Parachlorella, Crypthecodinium, and Schizochytrium comprises pulp and paper mill lignocellulosic hydrolysate with simplified sugars and one other non-sugar process residual also present in the hydrolysate to provide enhanced growth or faster fermentation cycle time.

In some embodiments, the culture medium contains process residuals from the wood lignocellulosic hydrolysis process that comprise non-sugar agents that are organic acids.

In some embodiments, the culture medium contains acetic acid as part of the lignocellulosic hydrolysate or as part of a microbial conversion solution wherein the microbe has produced the acetic acid from the lignocellulosic hydrolysate. In one embodiment, the acetic acid is present with at least one other fixed carbon source, for example, sugar. In a further embodiment, the acetic acid is always with at least one other fixed carbon source which is a sugar.

In one embodiment, the wood lignocellulosic sugar stream provides a slip stream that is used to make organic acid by microbial conversion (bioconversion). The lignocellulosic sugar is conveniently provided in the same solution as the resulting organic acid due to incomplete utilization (incomplete bioconversion) by the converting microbe, such as a bacterium.

In another embodiment, the invention provides a culture medium comprising a lignocellulosic sugar and a wood-derived organic acid, where the organic acid is the preferred fixed carbon source for one cell type and the sugar is the preferred fixed carbon source for the other cell type during heterotrophic or mixotrophic fermentation. In one embodiment, the algal culture medium can be supplemented with sugar from the lignocellulosic hydrolysate stream or from another sugar source. In one embodiment, lignocellulosic hydrolysate stream is used to stress an algal culture in the wood-derived organic acid medium.

Accordingly, the fixed carbon source used in the methods described herein can be a carboxylic acid, sugar acid, or chlorogenic acid. Non-limiting examples of a fixed carbon source include acetic, succinic, citric, fumaric, glycolic, malic, pyruvic, glucunoric, galacturonic, formic, levulinc, or proprionic acid. In certain embodiment, the organic acid used as a fixed carbon source can be derived from lignocellulosic biomass. Additional examples of a fixed carbon source are known to a person of ordinary skill in the art and such embodiments are within the purview of the invention.

In certain embodiments, the glucose to nitrogen ratio can be optimized to provide the best profile of a product.

In an even further embodiment, the invention provides a heterotrophic co-cultivation with at least one other microorganism. In an embodiment, the mutualism between two microalgal strains is described where accumulation of high levels of ammonium (NH₄⁺ [symbol] NH₃) or other metabolites that might otherwise inhibit cell division of one strain is mitigated by the other strain.

In certain embodiments, co-culture or co-cultivation is used as a strategy to promote proliferation of the target species. In a particular embodiment, co-culture is between a strain that requires organic acids as its fixed carbon source for heterotrophy and another strain that does not require organic acids for heterotrophy and can preferentially utilize ammonium or the other metabolite such as ethanol, lactate, or formate that can accumulate under low oxygen.

The present invention further relates to generating and cultivating microorganisms suited for heterotrophically producing high yields of carotenoids for biomass and products containing said microorganisms or said carotenoids.

In certain embodiments, a culture medium for a member of the genus Mayamaea comprises pulp and paper mill lignocellulosic hydrolysate with simplified sugars and one other non-sugar process residual also present in the hydrolysate. In one embodiment the microalgal cell of the genus Mayamaea expresses significantly reduced fucoxanthin pigmentation. Many diatoms are excellent sources of PUFAs and also characterized by containing fucoxanthin, which generally are extracted out with the lipids. Thus it is preferred to reduce pigmentation for facilitating the decolorization of the lipids.

In certain embodiments, heterotrophic cultivation of a genetically engineered organism is described. In one embodiment, a culture medium, for mixotrophic or heterotrophic fermentation of a member of the genus Chlamydomonas, consists of pulp and paper mill lignocellulosic hydrolysate that has been further processed by microbial conversion into a mixture comprising organic acid and residual unconverted sugars.

In another embodiment, a culture medium for a member of the genus Scenedesmus comprises pulp and paper mill lignocellulosic hydrolysate with simplified sugars and one other non-sugar process residual also present in the hydrolysate. In a preferred embodiment the microalgal cell expresses an added integrated heterologous gene.

The method described herein enables hydrolysate solutions to be easily manageable, provides a means to be cultivable with hardwood and softwood hydrolysates, production all during the year at a mill, and from which the desired product can be obtained economically in high yields.

The methods used in harvesting and further processing the biomass for isolating a product of interest are well known in the art. For example, some non-limiting methods of harvesting are centrifugation, flocculation, and filtration for dewatering. Some methods of extraction are in organic solvents, in edible oil, and by pressurized fluid and gas. In certain embodiments the heterotrophically produced biomass is used directly or as an admixture in animal and fish feed. For example astaxanthin-containing biomass is used for fish feed, and recombinant Chlamydomonas biomass is used in poultry feed. In other embodiments the pigments are extracted, for example astaxanthin is extracted as described in the U.S. Pat. No. 6,022,701. A myriad of applications for astaxanthin include those described in the art, for example, in Ambati et al. 2014, Tables 4 and 5.

A further embodiment of the invention provides methods for improved cultivation of cells under mixotrophic conditions. In some cases it is desirable that light be supplied to increase the growth rate of the cells beyond that of heterotrophic conditions. In H. pluvialis, the specific growth rate under mixotrophic conditions is 2.5 fold higher than the specific growth rate under heterotrophic conditions. In C. reinhardtii, the specific growth rate under mixotrophic conditions is 1.8 fold higher than the specific growth rate under heterotrophic conditions. The combination of a lignocellulosic sugar and a non-sugar agent in their unpurified form in a hydrolysate described in the present invention are well suited for mixotrophic systems. This may be particularly advantageous for increasing pigment accumulation beyond the already high levels seen under heterotrophic conditions, obtaining higher biomass yields, or shortening a production cycle time. A further advantage of mixotrophic growth is that dissolved oxygen levels in the culture medium will be easier to maintain as the cells will be producing oxygen as they fix CO₂ using light.

An embodiment of the invention provides methods of fermentation that do not require differentiation of the cultured cells for massive accumulation of a product of interest. Another embodiment of the invention also allows significant biomass, carotenoid, and lipid accumulation in the dark for measurably high specific growth and productivity rates to enable short cycle times. Thus, fermentation methods and cells are described that provide higher yields by a simple process in the dark without the need for cell differentiation. The methods of the invention provide culturing a microalgal cell, for example, a genetically modified algal cell, in a secure heterotrophic platform which transforms algal manufacturing for significant economic gain.

The methods of the invention provide:

1) higher production outputs, shorter incubation time;

2) simpler production logistics for feedstock by removing the step for fixed carbon purification;

3) altered product profiles;

4) faster industrial scaling by co-location at pulp and paper mills, with existing infrastructure useful for microbial fermentations;

5) use of a carbon feedstock with year round supply, at very large scale;

6) use of a carbon feedstock and process residuals with high efficiency of bioconversion, and with complete utilization of the glucose and xylose in a wood hydrolysate

7) significantly increased inventory and access to much larger markets using the wood-derived feedstock; and

8) large-scale economic production of multiple species, including those used as hosts for recombinant molecules under GMP and regulatory restrictions.

Examples of general principles and methods for heterotrophic algae cultivation, such as establishing axenic cultures, using a seed train with a plurality of passages prior to addition of final inoculum, the design of the fermentors that prevent illumination or add illumination of the microalgae, and cultivation until harvest or partial harvest, are described in the art, for example, in U.S. Pat. No. 8,278,090, which is incorporated herein by reference in its entirety.

In particular embodiments, the inoculum added to the fermentor can be produced by cultivation of the microalgae in the dark for at least one passage prior to its addition to the fermentor, or by prior cultivation in the dark for a plurality of passages, e.g., 2 passages, 3 passages, 4 passages, or 5 or more passages. In certain embodiments, after cultivation of the microalgae in the fermentor for a period of time in the dark, all or a portion of the microalgae can be transferred to a further fermentor vessel, where the microalgae can be further cultured for a period of time, wherein the further vessel prevents exposure of the microalgae to light. In practical terms, microalgae reported as having a mixotrophic capability, for example, various members of the Trebouxiophyceae, Bacillariophyceae, Eustigmatophyceae, Prasinophyceae, are candidates for the practicing of the invention.

Harvest or separations, biomass processing, handling of intact biomass as a product, cellular lysis, product extraction, supercritical fluid processing, or other isolation and purification of products may be done by using any methodology known to a person skilled in the art. Non-limiting examples of such techniques are described, for example, in U.S. Pat. Nos. 8,278,090 and 7,329,789, both of which are incorporated by reference. Non-limiting examples of product recovery include the separating different target compounds by use of a fractional distillation column. Further non-limiting examples for concentration, drying, powdering, grinding in preparation for extraction or use as a biomass for animal and fish feed, are described, for example, in U.S. Pat. No. 6,022,701 and EU Patent Application Publication No. EP1501937, both of which are incorporated herein by reference. US Patent Application Publication No. 20120171733 describes various means for cell lysis that are incorporated herein by reference.

US Patent Application Publication No. 20090214475, which is incorporated herein by reference, describes soft wall mutant strains of Haematococcus pluvialis for improving the extractability and bioavailability of natural astaxanthin, and their use in animal feed, human dietary supplements, pharmaceuticals, and foods. This can apply to other non-cyst forming species that product astaxanthin such as Monoraphidium, Scenedesmus and Chlorella.

Typical microbial growth curves or growth cycles are seen using a fermentor. As an example, an inoculum of cells when introduced into a medium is followed by a lag period before cell growth or division begins. Following the lag period, the growth rate increases steadily and enters the log, or exponential, phase. The exponential phase is followed by slowing of growth (cell division) due to nutrient depletion and/or increases in inhibitory substances. When growth stops the cells enter a stationary phase or steady state.

In certain embodiments of the present invention it is desirable to heterotrophically cultivate a genetically engineered microorganism, for example using synthetic biology where pathways are introduced or templates for hairpin nucleic acids are introduced, to enhance traits such as the production of a recombinant molecule, to modify the properties or proportions of components generated by the microorganism, or to improve or provide de novo growth characteristics.

Genetic engineering of Haematococcus spp. (Sharon-Goj man et al. 2015), Chlamydomonas spp. (Lauersen et al. 2013; Scaife et al. 2015; Scranton et al. 2015), Scenedesmus obliquus (Guo et al. 2013), Prototheca and Chlorella are well documented and incorporated by reference herein.

As such, a method for synthesizing a product of interest is provided herein. The method comprises the steps of:

providing a culture medium comprising wood-derived lignocellulosic simplified sugar in the presence of a non-sugar agent, wherein the non-sugar agent is a process residual of wood lignocellulose hydrolysis or an organic acid solution obtained by microbial conversion of wood-derived lignocellulosic sugar;

providing a microalgal cell that produces the product of interest;

culturing the microalgal cell in the culture medium to produce a microalgal culture from the microalgal cell; and

purifying the product of interest from the microalgal cells.

The product of interest can be a microalgal biomass comprising the microalgal cells, lipid, protein, amino acid, recombinant molecule, or a pigment.

The pigment can be a carotenoid that is an astaxanthin.

The protein can be a total crude protein or a peptide fragment of a protein.

The lipid can be a total crude lipid, a phospholipid, a fatty acid, or a long carbon chain polyunsaturated fatty acid.

The recombinant molecule of interest can be a heterologous protein or a nucleic acid.

Also, the methods described herein provide for the heterotrophic growth and synthesis of the product of interest occurs during the step of culturing, for example, vegetative growth under nutrient replete conditions for phospholipid and protein production, and stationary growth for polyunsaturated fatty acids, and wherein the step of culturing is performed under a fed-batch fermentation.

As mentioned above, unlike conventional methods where the product of interest is synthesized using purified sugars or organic acids, the methods provided herein permit the synthesis of a product of interest using unpurified sugars or organic acids to simplify the feedstock processing steps.

As mentioned above, unlike conventional methods where the product of interest is synthesized using culture medium with the sequential additions of different purified sugars (e.g., hexose followed by pentose), or sequential additions purified sugar and then organic acid, the methods provided herein permit the synthesis of a product of interest under simultaneous supply of the compounds for the cell culture.

Because these algae are produced in closed culture systems to exclude contamination, the final biomass is of high quality suited for a variety of novel animal and human uses. The closed fermentation systems also offer large quantities at lower cost, being produced at higher densities and faster growth rates within a short cycle time of merely days. As the carbon feedstock for the fermentations are sourced from wood byproducts of vast supply compared to the seasonal grasses or other agricultural wastes, the algal products can address markets of high volume much more readily than the other feedstocks.

In certain embodiments, the product of interest is altered in component composition, proportion, or temporal expression as compared to a control, wherein said control is a product of interest produced by culturing a microalgal cell expressing said product of interest in culture medium comprising the simplified sugar but not containing the non-sugar agent.

In certain embodiments, the product of interest is altered in component composition, proportion, or temporal expression as compared to a control, wherein said control is a product of interest produced by culturing a microalgal or microbial cell expressing said product of interest in culture medium comprising non-lignocellulosic sugars.

In certain embodiments of the culture method of the invention, the product compositional analysis differs substantially from that seen by conventional methods.

In certain embodiments of the culture method of the invention, without yet undergoing optimization, the product compositional analysis can be as good as or better than the best composition achieved with conventional methods.

In certain embodiments of the culture method of the invention, on a volume basis, the dry cell weight of the microalgae is greater than the dry cell weight of the same strain of microalgae cultured with a purified fixed carbon source that would require further processing steps to obtain and with all other culture conditions being the same. The dry cell weight of the microalgae grown using the culture medium of the invention can exceed the dry cell weight of microalgae grown using the same hexose and pentose source by at least: about 40%, about 80%, about 100%, about 120% or more, or by an amount within any range having any of these values as endpoints.

The step of isolating and purifying the product of interest may comprise one or more steps of drying, grinding, lysing, or extracting the microalgal cell.

The step of culturing can be performed under mixotrophic conditions, at least for a portion of the culturing step.

The following examples are provided to describe the invention in further detail. These examples serve as illustrations and are not intended to limit the invention.

### EXAMPLE 1

### Wood Hydrolysate from Pulp and Paper Mill Material, Microbial Species and Fermentation Conditions

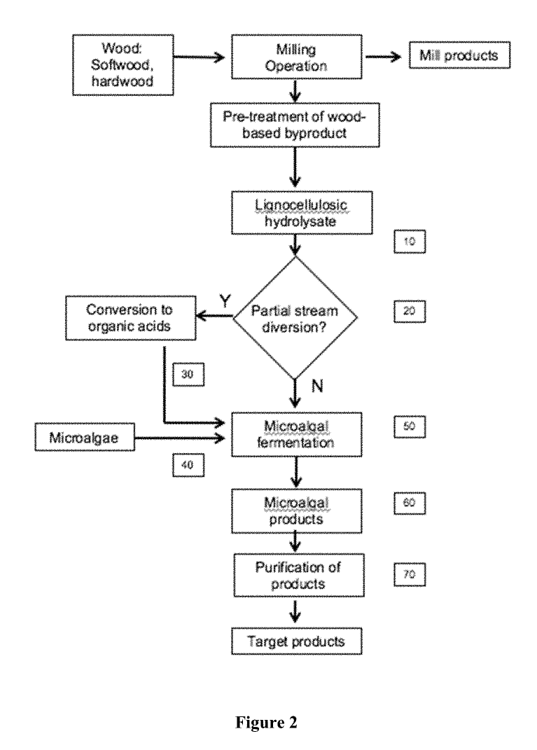

Enzymatic hydrolysates of various compositions are produced courtesy of Cellulose Sciences International (Madison, Wis.) and Domtar International according to U.S. Pat. No. 8,617,851 from various woody biomass, supplied by Domtar International, subjected to alkali plus co-solvent pre-treatment (Table 5). The enzymes product, used according to manufacturer's direction, was Cellic Ctec2 (Novozymes) that is a blend of cellulases, beta-glucosidases, and hemicellulase. Incubation was 72 hours with agitation, 50° C., solids loading of 2%, followed by filtration through a 10 kD filter to remove the enzymes. Lignocellulosic hydrolysates (FIG. 2, [10]) from softwood and hardwood were prepared and analyzed. The algae strains selected for testing are based on their potential biomass applications for biofuels (lipids), feed (whole biomass, protein and lipids), and specialty products (colorants, nutritional lipids, emulsifying lipids) and capable of heterotrophic or mixotrophic growth. These include Hawaii-collected Chlorella and Scenedesmus identified at the species level based on 18S sequence DNA sequencing, as described in Kuehnle et al. 2015: KAS908 is 100% identical to Chlorella sorokiniana; KAS740 is 100% identical to Scenedesmus armatus. Other non-limiting strains are listed elsewhere in the examples. Cultures were screened previously for their ability to grow on sugars and were adapted for heterotrophic growth in modified F/2-Si (Si-free) fresh water medium containing 18 g/L glucose plus 1.8 g/L yeast extract (YE). Basic recipes of F/2 and F media (detailed in Guillard 1975; Guillard 1962), contain all the nutrients essential for growth of many fresh water microalgae and are easily modified by omission of seawater and silicates. Use of this medium is not limiting for the purposes of this invention. The pH of the hydrolysates ranges from 4.9 to 5.5, thus the pH of each medium containing wood hydrolysate is adjusted to pH 7.0 using 1M TRIS-HCl (pH 8.0) prior to inoculation. Wood hydrolysates are initially tested for growth at small scale and the wood hydrolysate concentration with highest growth for each strain was identified. Briefly, heterotrophically adapted KAS908 and KAS740 are grown in 96-well plates on an orbital shaker 100 rpm using wood hydrolysate standardized to 18 g/L and 9 g/L total sugars along with the components that comprise modified F/2-Si fresh water plus YE medium, 26° C. These strains are further grown in 50 ml medium in 250 ml shake flasks on an orbital shaker 100 rpm at suitable wood hydrolysate concentrations found during small-scale tests. Growth is monitored daily by measuring OD750.

For Crypthecodinium cohnii (ATCC 307727; KAS1701) cells of the obligate heterotrophic DHA producer are grown in medium with 20% or 40% BSP hydrolysate (9 g/L or 18 g/L total sugars), 1.8 g/L yeast extract (Difco, Sigma-Aldrich), and 60% seawater (equivalent to 21 g/L sea salt), pH 6.5. KAS1701 is cultured in 50 ml medium in 250 ml shake flasks on an orbital shaker 100 rpm at 26° C. in the dark. For Schizochytrium limacinum SRI (ATCC MY A-1 KAS1707), cells of the obligate heterotrophic DHA producer are cultured (26° C. in the dark at 100 rpm) and adapted to ½ strength seawater (17.5 g/L Instant Ocean) medium containing 25 g/L glucose supplemented with yeast extract, trace elements, and vitamins as described (Ren et al. 2009). Then 50 mL of log phase culture is sub-cultured to a 450 mL volume SPBK hydrolysate diluted such that total sugars starts at 25 g/L (also contains 2.3 g/L acetic acid) plus nutrients in 1 L flasks.

Another strain tested is Rhodotorula glutinis (ATCC 2527; KAS1101) a red yeast with high protein and oil, capable of synthesizing β-carotene, torulene, and torularhodin, of interest as natural food colorants. It has shown synergistic effects when co-cultivated with Chlorella for increased biomass yield. Rhodotorula is maintained in YPD medium comprised of 10 g/L yeast extract (AMRESCO, VWR), 20 g/L, peptone (BD Bacto Peptone, Fisher Scientific), and 20 g/L glucose (Sigma-Aldrich). Other culture media, for growth comparisons, comprised 3% to 60% SHC hydrolysate with 10 g/l yeast extract and 20 g/L peptone (YP), with the resulting glucose concentrations: 5 mM (2.7 g/L glucose), 25 mM glucose (4.5 g/L 50 mM (9 g/L) and 100 mM (18 g/L) glucose.

TABLE 5
Composition of Softwood and Hardwood Enzymatic Hydrolysates.

| | | | | | | | Total | | | |
|---|---|---|---|---|---|---|---|---|---|---|
| | Total | | | | | | process | % Organic acids | | % |
| Hydrolysate from | sugars | % C6 | | | % C5 | | residuals | Acetic | Lactic | Alcohol |
| Wood | (g/L) | Glu | Man | Gal | Xyl | Ara | (g/L) | Acid | acid | Ethanol |
| Hardwood HWD | 18.68 | 60.52 | 1.51 | 1.79 | 35.60 | 0.63 | 5.11 | 2.15 | 52.52 | 45.32 |
| (similar to SHC) | | | | | | | | | | |
| SPBK (Southern Pine | 87.87 | 89.44 | 0.17 | 2.04 | 6.83 | ND* | 8.09 | 100 | ND | ND |
| Bleached Kraft) | | | | | | | | | | |
| SPFC (Southern Pine | 21.51 | 62.00 | 18.14 | 0.78 | 18.44 | 0.65 | 3.67 | 50.16 | 49.94 | ND |
| Finer Chips) | | | | | | | | | | |
| BSP (Bleached | 44.33 | 90.91 | ND | ND | 9.09 | ND | Not Provided | | | |
| southern Pine) | | | | | | | | | | |
| SHC (Southern | 27.50 | 56.96 | 0.69 | 13.02 | 29.33 | ND | ND | ND | ND | ND |
| Hardwood Chips) | | | | | | | | | | |

Hydrolysates profile: pH: 5.5

*ND: not detected.

Heterotrophic growth experiments are performed in the dark for wood hydrolysates at a larger scale using a 10-L BioFlo110 fermentor (New Brunswick Scientific, Enfield Conn.) and pre-established batch fermentation conditions of T=30° C., pH=7.0, agitation=300 rpm, DO=100%, and air=7 L/min. Briefly, KAS908 is inoculated to a density of 2 g/L in fresh water medium, equivalent to 2× the concentration of F/2 medium, comprised of wood hydrolysates standardized to 18 g/L total sugars and the components of F medium (0.2 g/L Cell-HI F2P, Varicon Aqua Solutions, Worchestershire UK) plus 1.8 g/L yeast extract.

Samples are collected every 24 hours for five days and analyzed for biomass growth measurement (dry weight), as well as for glucose and xylose utilization through HPLC. Culture samples in 25-mL quantities are collected and immediately centrifuged at 3,000 rpm. The supernatant from each sample is analyzed for glucose and xylose by HPLC using a Waters 2695 Alliance Separations module with a Rezex RPM-Monosaccharide Pb+2 (8%) column (Phenomenex, Torrance, Calif., USA) and a 2416 refractive index detector (Waters Corp., Milford, Mass.). Samples not immediately analyzed are stored at −20° C. until further use. The system is run isocratically with deionized ultra-pure water. The injection volume is 40 μL/min with a 20 min run time at 85° C.

Nitrate concentration is monitored qualitatively using a nitrate test kit (Aquarium Pharmaceuticals, Chalfont, Pa.). As positive controls and to establish baseline kinetics, fermentation using mixed C5 and C6 model sugars is also performed. In some cases, KAS908 is grown in F medium (modified for fresh water) containing 16.34 g/L glucose and 1.66 g/L xylose plus 1.8 g/L YE to mimic the corresponding hydrolysate from a first batch of Bleached Southern Pine and grown under the same batch fermentation conditions for five days. BSP is identified as similar to SPBK by the supplier of the hydrolysate, and made available in a subsequent preparation for additional larger scale experiments. Biomass productivities (g/L/day) and biomass yield on sugar (g total biomass/g sugar utilized) are calculated. Additional analytical methods utilized are described in the other examples.

Cells from the 10 L volume of KAS908 fermentation culture can be used to directly seed a 80 L volume (10 L culture+70 L fermentor heterotrophic media in an Eppendorf BioFlo 610 fermentor). The 80 L culture is fed nutrients using automated peristaltic pumps using BioCommand software and pH is maintained at 7.5 with 0.1 M NaOH and 0.2 M H₃PO₄ as needed. The sparged air at 50-100 LPM and Rushton blade agitation to 350 rpm or higher are controlled by a cascade and are increased as dissolved oxygen in the system drops below 50%. By this method the resulting biomass (16 g/L from an initial 0.2 g/L) is produced over 96 hours that includes no lag phase and a 72-hour extended logarithmic phase of high specific growth of 1.4/day. For this and other species, it is understood that scaling from about 100 L to 1000 L to 100,000 L vessels and such can proceed using the basic conditions modified for mass balance, aeration, viscosity and cycle time as is known in the art.

The availability of differing preparations of feedstock informs a strategy for the carbon feed during the fermentation cycle, as the microalgal density increases and fermentation reactor capacity becomes more limiting; and for the choice of microalgae and co-cultivation option (if it prefers wood-derived 2-, 3-, 5-, and 6-carbon feedstocks derived from lignocellulosic biomass). The production volume is comprised of relatively dilute hydrolysate at the outset. As the culture growth actively increases, the carbon is proportionally supplied from conditioned, concentrated hydrolysate stream with minimal impact on working volume. In general terms, a concentrated feedstock facilitates high microalgal cell densities with minimal impact on working volume. This is followed by a finishing stage for the product of interest, as is known in the art. For example, N stress or cold stress, are used to promote carotenogenesis (for pigment accumulation) or lipogenesis (such as for omega 3-, 6- and 9-fatty acids accumulation), as shown in subsequent examples with several species and co-cultures. It is also understood that strains can be selected for improved product yield from populations cultured on wood hydrolysates, such as from various sources and concentrations, for increased productivities over time.

### EXAMPLE 2

### Biomass Production on Wood Hydrolysates

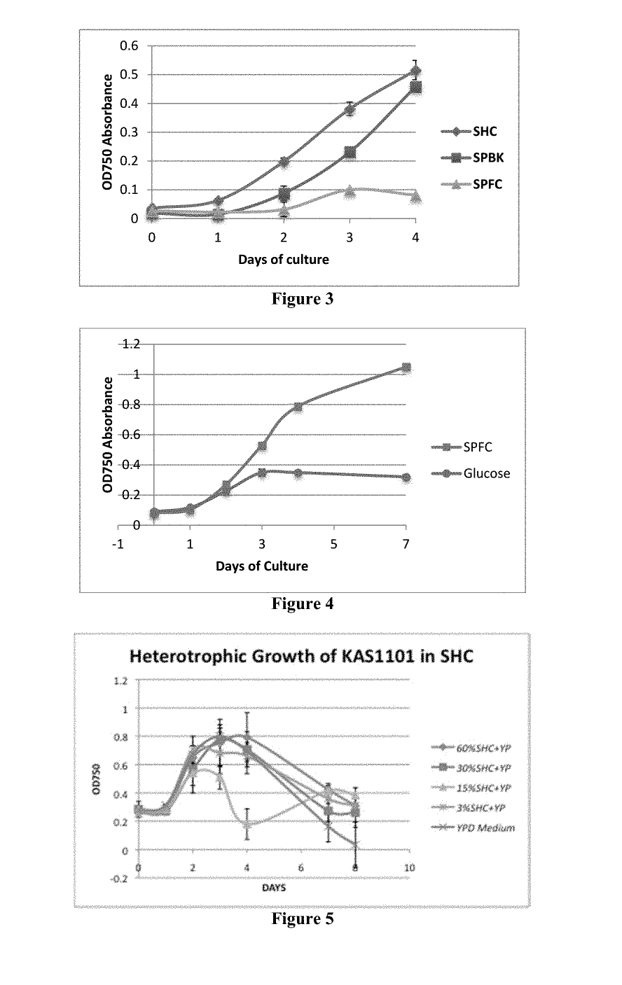

Multiwell plates are used as an initial screening tool to determine the capability of microalgal cultures to grow in the dark on wood hydrolysates from pine softwood, southern hardwoods and northern hardwoods. Surprisingly, all wood enzymatic hydrolysates tested support growth and biomass production of microalgae, though performance varies with each type of hydrolysate. For example, the three wood hydrolysates designated SHC, SPBK, and SPFC (Table 5), standardized to 18 g/L total sugars, show different growth profiles for Chlorella KAS908, with one hydrolysate (SPFC) being inhibitory for the first four days (FIG. 3). During this period, culture using SPFC in the dark shows nominal growth (OD750 between 0 and 0.1) similar to the negative control in the dark using F/2 with yeast extract and no added sugars or hydrolysate (OD750 between 0 and 0.1), while the growth of positive controls on 9 g/L glucose and 18 g/L glucose reaches OD750 above 0.3 by day 3.

Surprisingly, softwood and hardwood hydrolysates produce similar performance. The softwood (Southern Pine Bleached Kraft) yields active growth similar to hardwood (Southern Hardwood Chips), to reach only slightly less biomass yield by the fourth day although it has a longer growth lag for the first two to three days (FIG. 3, showing day 0-day 4 growth). Unexpectedly, onset of growth of KAS908 on Southern Pine Finer Chips (SPFC) is evident by Day 7, indicated by an increase in OD750 from less than 0.1 on day 4 to about 0.25 on day 7, whereas the negative control continued to show no growth as expected (OD750 less than 0.1); the positive control on 18 g/L glucose neared OD750 of 0.4 by day 4. These data indicate a strategy of acclimation to certain hydrolysates to mitigate inhibition or possible inhibition by the process residuals, enabling use of higher amounts or concentrations of hydrolysates.

Surprisingly, Scenedesmus KAS740 cells can utilize process residuals. Using KAS740 grown in flasks, use of SPFC corresponding to 9 g/L total sugars grows better (60% higher OD value at time of glucose depletion) than in medium containing 9 g/L glucose alone based on Student's t means testing (p=0.02; FIG. 4). Process residuals of Southern Pine Finer Chips contain two organic acids, acetic acid and lactic acid, while Southern Pine Bleached Kraft contains acetic acid and no lactic acid.

A red yeast, Rhodotorula glutinis, KAS1101 is grown in 96-well plates using various concentrations of SHC hydrolysates to compare with YPD medium with 20 g/L glucose. As shown in FIG. 5, KAS1101 growth is uninhibited in the medium employing the highest amount tested of 60% SHC. It shows the same growth as the control YPD medium on Day 2 and superior growth as the control by Day 4, with extended biomass yield for one additional day based on Student's t means testing (p=0.006; FIG. 5) due to the process residuals.

A simple screen for relative growth patterns of microalgal species such as described here can be used to assist mills, which may be limited to producing a particular wood hydrolysate based on the mill products. Depending on their target products of choice, the mill may decide for conversion of a slipstream of hydrolysate into a second carbon feedstock (FIG. 2, Y), such as into acetic acid, to then support microalgal bioconversion using species that favor organic acid as the primary fixed carbon source. See Example 5.

### EXAMPLE 3

### Biomass Product, Some Extractives, and Sugar Conversion Efficiency Using Wood Hydrolysates

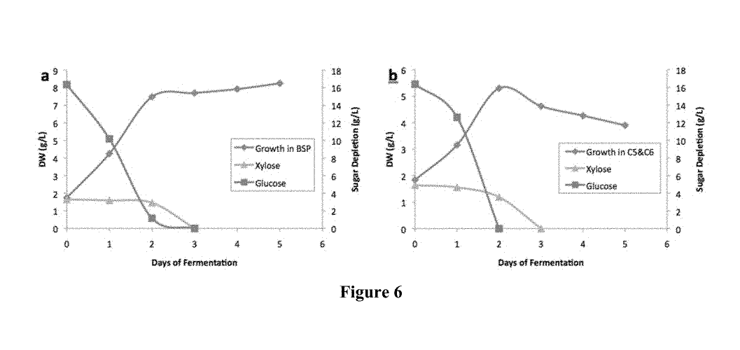

This example demonstrates higher biomass productivities on wood hydrolysate than on model sugars and higher than expected efficiency of bioconversion. Growth of Chlorella KAS908 in a medium based on softwood hydrolysate, Bleached Southern Pine (BSP with 2F+1.8 g/L YE) hydrolysate, is compared to that in a medium containing an equivalent mixture of C5 and C6 model sugars (16.34 g/L glucose and 1.66 g/L xylose) using a 7-L dark stirred fermentor. Surprisingly, the wood hydrolysate with monosaccharides and process residuals outperform the model sugars alone, with a 1.6-fold (160%) higher biomass productivity of 2.87 g/L/day compared to 1.7 g/L/day for the control Chlorella. KAS908 utilizes the glucose and xylose in series during dark fermentation, as shown by a decrease and eventual complete depletion of both sugars in the culture medium containing wood hydrolysates (FIG. 6a), a feature mimicked during growth on model sugars (FIG. 6b).

Higher biomass production for both DHA-producing C. cohnii KAS1701 and Schizochytrium KAS1707 is observed using hydrolysate compared with using model sugar in batch fermentation flasks per conditions in Example 1. KAS1701 grown in BSP wood derivative, corresponding to 9 g/L total sugars plus 1.8 g/L YE, shows rapid increase in OD750 from 0.5 to 4 on day 2 and 1.4 times higher yield than on pure glucose (OD750 from 0.5 to 3) before reaching glucose depletion. Lipid and fatty acid analysis indicates a lipid content of 13% DW, with DHA comprising 30% of the lipid fraction for 4% DW. Much higher productivities are obtained when supplied with non-limiting feedstock in fed-batch mode and sufficient aeration. Switching to distillation-purified ligoncellulosic acetic acid (such as used in Example 5) also yields DHA, at 9% DW. For KAS1707, the control flask (glucose as only carbon source) and the SPBK flask the initial biomass of 0.5 g/L grows to 8 g/L and 9.2 g/L, respectively, in 72 hours with a maximum specific growth rate of 0.95/day and 1.1/day respectively. The cells are allowed to accumulate lipids for an additional 24 hours and are harvested at 96 hours after inoculation. Volumetrically the SPBK grown biomass contains 1.84 g/L (20% of the total biomass) total fatty acids and 0.46 g/L (5.0% of the total biomass) of the fatty acid DHA. The control flask contains 1.44 g/L (18% of total biomass) total fatty acids and 0.37 g/L (4.6% of the total biomass) of the fatty acid DHA. The lipids are 1.1 times higher and the DHA is 1.08 times higher in the presence of process residuals from the softwood hydrolysate. With no carbon or nitrogen limitation for the purposes of reaching higher densities on SPBK concentrated to allow 140 g accumulative hexose during the course of fed-batch fermentation, cultures reach 70 g/L biomass with 19 g/L (27% of total biomass) fatty acids. Higher biomass productivities translate to increased biomass product yield and shorter fermentation cycle times. Even higher final omega-3 fatty acids can result under nitrogen deficiency.

Biomass yield on sugar consumed (dry weight of biomass produced per gram of sugar utilized) is also determined, as a parameter useful in calculating overall process efficiency and biomass production cost. Results show that sugar utilization of microalgae, using different hydrolysate streams, varies with the composition and impurities present in them. Surprisingly, a high bioconversion ratio of 1.15:1 biomass produced per gram of sugar utilized, as measured by HPLC, is obtained for KAS908 grown in the hardwood hydrolysate, SHC. This exceeds the theoretical biomass yield per gram sugar utilized of 0.5:1 reported for protein-rich algal biomass ideal for animal feed, and is attributed to the assimilation of process residuals in the hydrolysate; the hydrolysate glucose and xylose are completely depleted. The hardwood preparation has a relatively high ethanol content, along with several organic acids, and hydrolysate is known to contain furfurals. Also, BSP hydrolysate gave a 0.45:1 ratio, close to the theoretical biomass yield per sugar utilized. The development of this method that proves suitable for microbial growth using cellulosic hydrolysates from softwoods, especially from Southern Pine, known for their unique toxic fermentation inhibitors, is beneficial to help advance implementation of the pulp and paper mill biorefinery concept.

These outcomes using the method of the invention show a high compatibility of different algal genera for heterotrophic growth on wood hydrolysates and the ability to scale-up. This is required in order to develop process economics of using microalgae for target products, such as protein, lipids, and pigment production; to select host strains for recombinant product production that will be compatible with a certain mill's lignocellulosic feedstock; as well as to contribute to the potential of establishing integrated biorefineries for the pulp and paper industry. Once the target algal products are identified for a mill, the algal strain can be optimized in tandem with the sugar and nutrient feeds as well as operational conditions. Some examples of other target algal products and alterations follow.

### EXAMPLE 4

### Algal Biomass Quality for Value-Added Products

In the next two examples, production of four different product classes, are exemplified through the use of microalgae cultured in wood hydrolysates. These comprise lipids, protein, pigments, and recombinant product. It is understood that these are non-limiting examples, and that the production process can be optimized for each cell type to provide a preferred duration of the production cycle and preferred culture conditions throughout the fermentation to achieve the desired product formation.

A glucose:nitrogen ratio screening is performed to determine preferred ratios for improved quality of algal biomass for value-added products. Heterotrophically acclimated KAS908 cultures are grown in shake-flasks in a medium (2F+YE) with the following glucose:nitrogen ratios (w/w): 1:1, 3:1, 4:1, 5:1, 6:1, 9:1 and 13:1. On day 3, the medium with 13:1 ratio gave the highest biomass density by (OD750 of 1.2) and the medium on 1:1 ratio gave the lowest biomass density (OD750 of 0.4). Total crude fat is determined by acid hydrolysis/petroleum ether method (AOAC 954.02 by New Jersey Feed Labs, N.J.) and expressed as a ratio per total soluble proteins. Alternatively, lipids are extracted and assayed in algal cells using modified sulfo-phospho-vanillin methods (Cell Biolabs Lipid Extraction and Quantification Kits, San Diego, Calif.) following manufacturer instructions. Protein is extracted using a modified standard method for algae by Rausch (1981) and quantified using the Bradford reagent (ThermoFisher), with absorbance measured at 595 nm using a GENESYS 10S UV-VIS spectrophotometer. Relative amino acids are determined by AOAC 994.12 and 985.28 by New Jersey Feed Labs. Analysis of phospholipids is by thin layer chromatography (TLC). Biomass was extracted in 2:1 chloroform:methanol (20 μL per mg biomass) and volumes from extractions were loaded onto TLC lanes after being normalized on a lyophilized-dry weight basis. Plates (Sigma-Aldrich TLC plates silica gel matrix, Sigma-Aldrich Co.) are run in a TLC chamber with chloroform:methanol:water 65:25:4 for 20 minutes, dried for 5 minutes, then sprayed with molybdenum blue spray reagent (Sigma-Aldrich Co.) and developed for 1 hour.

For pigment analysis, dewatered samples are pelleted by centrifugation at 3000 g for 5 minutes, frozen at −80° C., and freeze dried to determine dry weight. Pigments are extracted from ground freeze-dried biomass with 50 μL of acetone per mg of biomass for 5 minutes at room temperature. For astaxanthin, mean pigment in acetone is determined by calibrated spectrometry using the A476 absorbance adjusted by the extinction coefficient of astaxanthin (217) and proportion of total carotenoid that is astaxanthin in the vegetative cell type (75%).

Constitutive (i.e., from the growth phase, non-stationary) protein and lipid ratios are compared between biomass grown in 40% BSP hydrolysate with F nutrients and the biomass grown in the dark in F medium with equivalent amounts of 18 g/L total sugars (glucose+xylose basis). Biomass derived from the hydrolysate culture contains an altered protein to lipid ratio of 1.8:1 compared to 3.4:1 for the heterotrophic control on glucose and xylose sugars alone. A more lipid-rich biomass on hydrolysate—under the conditions used—is advantageous for products of extracted oils such as for omega-3 fatty acids, EPA and DHA, and for phospholipids. Addition of organic acid-rich hydrolysate or wood-derived organic acids (FIG. 2 [30]) can further increase the lipid content of the biomass of KAS908 in F with 18 g/L total sugars when under nitrogen-deplete conditions. Regarding altered profile of amino acids compared on a protein basis, biomass from the BSP hydrolysate has almost 140% higher methionine+lysine than the control biomass, and 50% lower valine. This can benefit poultry feeds, formulated on amino acid requirements, to which methionine+lysine is normally added. Dried whole algal biomass as a feed ingredient delivering protein and dried lipid (to replace a certain amount of oil added in the broiler diet) renders an improved quality pellet for feed formulators. In the case of Chlorella KAS908 grown under these conditions, biomass from hydrolysate shows slightly lower linoleic acid content than the control biomass (37% vs 39% relative basis), with lowered levels preferred for broiler performance. Results also show altered alpha-linolenic acid, with a decrease from 2.7% to 1.9% relative basis; ALA is added to animal feed to stimulate physiological functions.

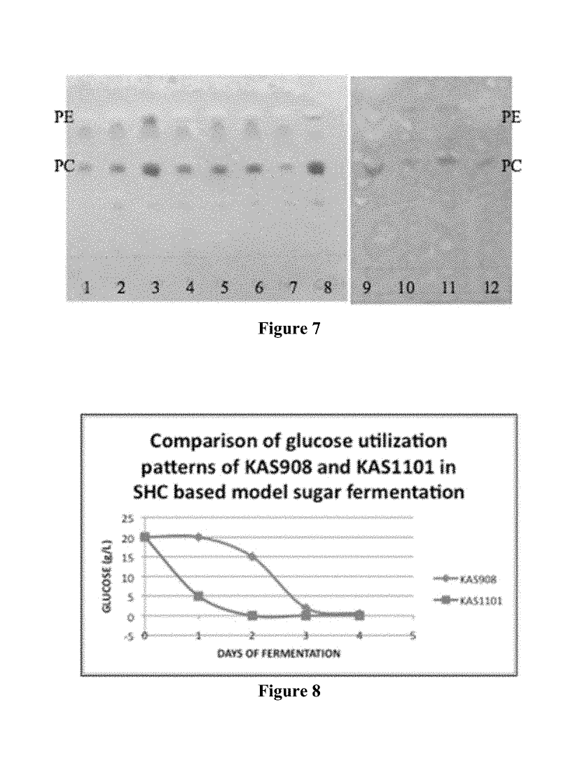

Qualitative results from TLC indicate that biomass grown for three days on the 4:1 ratio (Glucose:Nitrogen) gave the most intense bands for both PC (phosphatidylcholine) and PE (phosphatidylethanolamine) (FIG. 7). This is validated at fermentor scale carried out using the previously determined growth parameters (temperature=30° C., pH=7.0, DO₂=100%, agitation=300 rpm, air=3.0 L/min). KAS908 is grown in 2F medium containing a 4:1 ratio of glucose to nitrogen (e.g., 3 g/L glucose to 0.736 g nitrogen). Nitrogen sources are YE and Cell Hi (0.7 g/L YE, 0.3 g/L Cell Hi). A TLC chromatogram indicates that KAS908 grown in the dark for 3 days in a medium with 4:1 ratio of glucose to nitrogen supports higher production of phospholipids relative to the reference medium 2F+36 g/L glucose (FIG. 7, lane 3). This is compared to a 6-L dark fermentation on 40% Bleached Southern Pine wood hydrolysate (BSP) at 18 g/L total sugars. KAS908 biomass grown with BSP hydrolysate (FIG. 7, lane 11) yields only slightly lower TLC band intensities for phospholipids PC and PE than the best performing biomass grown on 4:1 glucose to nitrogen ratio medium (FIG. 7, lane 9). Biomass grown at higher hexose and pentose concentration (19.43 g/L glucose and 8.06 g/L xylose in 2F, FIG. 7, lane 10) has inferior yields, similar to 2F+36 g/l glucose (FIG. 7, lane 12). This shows that wood hydrolysate, specifically of Southern Pine, can be used directly (after dilution) for obtaining, for example, a target lipid product, with yields under unoptimized conditions using hydrolysate approaching the highest yields achieved under optimized culture using control medium without hydrolysates and without process residuals.

### EXAMPLE 5

### Algal Products Using Wood Lignocellulosic Sugar with Wood-Derived Organic Acids, Including Co-Culture

This example employs strains selected for preferred growth using organic acid under heterotrophic or mixotrophic conditions for two types of products, pigments and recombinant nucleic acids such as dsRNA or recombinant protein products. It is exemplified, but not limited to, using Haematococcus pluvialis and Chlamydomonas reinhardtii in dark cultivation, used alone as monocultures or in combination as co-cultures; as well as using Haematococcus pluvialis with a second cell type other than Chlamydomonas; the latter is exemplified but not limited to Scenedesmus obliquus, KAS1003, a Hawaiian accession previously confirmed by DNA fingerprinting as described (Kuehnle et al. 2015).

Southern pine lignocellulosic hydrolysate is overlimed and then further pH-adjusted with sulfuric acid to pH 5 prior to use for bacterial fermentation for bioconversion of sugars to acetic acid as known in the art (Mohagheghi et al. 2006), for example using Moorella thermoacetica ATCC 39073 (Clostridium thermoaceticum) according to Ehsanipour et al. (2016). The resultant solution, sustained at pH 6.8, contains about 1% unconverted lignocellulosic simplified sugars (glucose and minor C6 sugars) in the presence of 2% wood-derived acetic acid. For the purposes of algal culture, a portion of the 2% wood-derived acetic acid/1% wood-derived glucose is diluted 33.3 to 0.06% acetic acid (10 mM acetic acid) and 0.03% glucose (1.65 mM glucose) in growth medium (F with nitrate replaced with equal molar urea and 1/10^{th} yeast extract by weight of total carbon sources present), pH adjusted to 7 and filter-sterilized by 0.2-micron cross flow filtration to supply the initial acetic acid to start the fermentation. Also a portion of the 2% wood-derived acetic acid is concentrated 5× such as by distillation as known in the art to 10% acetic acid/5% glucose (or greater), pH 4, to supply carbon throughout the algae fermentation run. It is understood that higher acetic acid concentrations or purified slipstreams allow smaller volume increases in the fermentation tank and is preferred for very high cell density cultures. Alternatives include use of a multistep process to generate acetic acid at desired concentrations, such as with Acetobacter and prior ethanol conversion by Saccharomyces as is known in the art, as is using other efficient mutants of homoacetogens for a one-step process. Fungal species in addition to bacterial species are known in the art to produce high amounts of organic acids; notable genera include Aspergillus and Rhizopus.

The microalgal species are Haematococcus pluvialis KAS1601 (an improved strain of H. pluvialis UTEX 2505, Culture Collection of Algae at the University of Texas Austin, Tex., USA) and Chlamydomonas reinhardtii KAS1001 (137C, Chlamydomonas Resource Center, St. Paul, Minn. USA). These are maintained heterotrophically in 0.06% wood-derived acetic acid/0.03% wood-derived glucose medium and then transferred to preferred growth media for heterotrophic culture on acetic acid, using media as described in U.S. Ser. No. 62/356,896, with sodium acetate replaced with 0.06% wood-derived acetic acid/0.03% wood-derived glucose, adjusted to pH 7. The fermentation uses a 2.3 L fermentation vessel (New Brunswick BioFlo 115) at 1 L operated using BioCommand software with peristaltic pumps, and head plate ports. The pH is maintained at pH 7.7 to 7.3 for the duration of the fed-batch fermentation with pH-triggered additions of the concentrated 5× to 10% acetic acid/5% glucose (or greater), pH 4, and other inputs are monitored and maintained as described in US Serial No. 62/356,896. Carbon (10% wood-derived acetic acid/5% wood-derived glucose) is supplied throughout the fermentation run from 75 μL/L per hour up to 1500 μL/L per hour or more as the culture density increases.

The sole sources of fixed carbon inputs are the unconverted lignocellulosic simplified sugars and the bioconverted wood-derived acetic acid. For Chlamydomonas reinhardtii KAS1001 cultivated as a monoculture in fermentation over 120 hours, an initial 0.4 g/L algal culture produces a biomass with density of 6.5 g/L (specific growth rate 0.57/d). This is the first instance of biomass of this genus being cultivated in wood-derived feedstocks. Subsequently a C. reinhardtii KAS1402 is plastid-transformed as known in the art to carry an inverted repeat for a mosquito 3-HKT gene fragment per U.S. Ser. No. 62/356,896. A selected KAS1402 event that carries the 3-HKT dsRNA coding sequence when cultured under heterotrophic conditions on acetic acid or in combination with lactic acid reaches cell densities of 30, 50, and 85 g/L. A BioFlo 610 model 120 L vessel containing 90 L of media is fed nutrients as required for growth via automated feeding of nutrient concentrates; carbon feed and the pH of the culture is maintained between 6.9 and 7.6 using a 20% acetic acid concentrate. Oxygen is supplied by agitation at 500 rpm with 100 lpm gas flow with pure oxygen supplying up to 50% of the total gas flow. It is also shown that the Chlamydomonas grows on the wood-derived organic acid stream alone, in the absence of simplified sugars, to similar yields. This productivity greatly surpasses what is known in the art for using acetate including: ammonium acetate, sodium acetate, or potassium acetate.

For H. pluvialis, cells redden as ammonium concentration increases (2.5 mM and above). To mitigate the ammonium build-up during biomass generation, Scenedesmus obliquus KAS1003 is co-cultured at low density with the H. pluvialis KAS1601, as described in U.S. Ser. No. 62/356,896. The initial C6 sugars (0.3 g/L) needed by KAS1003 are supplied initially by the 33.3× dilution of 2% wood-derived acetic acid/1% wood-derived glucose as described above. Carbon (10% wood-derived acetic acid/5% wood-derived glucose) is supplied throughout the fermentation run from 75 μL/L per hour up to 1500 μL/L per hour or more as the culture density increases to supply both acetic acid and glucose. The initial ratio of KAS1003 to KAS1601 is such that KAS1601 produces more ammonium than the KAS1003 can consume so the ammonium concentration reaches >2.5 mM by 96 hours of fermentation (or, glucose and nutrients except urea and phosphate can stop being fed at 72 hours which allows the ammonium to reach >2.5 mM by 96 hours). The culture is allowed to ferment for an additional 24 hours to increase the astaxanthin content of the motile KAS1601 cells. For Haematococcus KAS1601 in unoptimized fermentation over 120 hours, an initial 0.2 g/L algal culture produces a biomass with density of 3 g/L, 1.2% to 2% pigment and 45% to 50% protein content. Base, unoptimized, usage of feedstock is about 4.2 mL glacial acetic acid equivalents required for every gram algal biomass using H. pluvialis. A preferred compositional profile for use of the intact biomass for aquaculture feed is possible by selecting the finishing step of urea or sulfate stress to obtain corresponding protein and pigment content desired by feed formulators and end users. A KAS1601 and KAS1003 (S. obliquus) fermentation with an initial cell density of 2 g/L produces 32 g/L biomass in 120 hours; an initial 3 g/L produces 48 g/L in 120 hours with 1.2% pigment and 45% protein with vegetative culture under sulfate stress. Agitation is with a pitched blade impeller at 350 rpm with gas flow at 1 vessel volume per minute and pure oxygen supplied as needed to maintain dissolved oxygen at >50%.

It is understood that the process can be optimized for each cell type and to select a preferred duration of the production cycle while achieving product formation, and to select a preferred compositional profile for target market use. In this example, it is understood that the S. obliquus can be interchanged with a different microbial cell type suited to heterotrophic growth as long as it still prefers a fixed carbon source that is not an organic acid, preferentially glucose or xylose, and preferentially consumes ammonium as nitrogen source, as is known in the art for many such cell types. Options among astaxanthin or other pigment producing cell types, or for oil-producing or high beta-glucan-producing cell types, are other species of Scenedesmus, Chlorella, Auxenochlorella, Monoraphidium, Euglena, Rhodotorula, and many different diatoms such as Phaeodactylum and Cyclotella, and thraustochytrids or thraustochytrid-like cell types, as known in the art.

Alternatively, ammonium control proceeds using H. pluvialis co-culture with Chlamydomonas reinhardtii, per U.S. Ser. No. 62/356,896. The final biomass is comprised of about 99% H. pluvialis biomass, similar to what may occur naturally in an open pond with mixed microorganisms. Adjustment of co-cultivation parameters such as dosing of the cell types allows reaching different target rates of growth and productivity relative to the carotenogenesis trigger for H. pluvialis of about 2.5 mM ammonium.

It is understood that the process can also be optimized for the composition of the hydrolysate that is produced, depending on the hydrolysis process and type of wood processed by any particular mill, and the degree of dilution/concentration of the hydrolysate. Some compositions and profiles are known in the art, examples of which are described by Burkhardt et al. (2013); Brodeur et al. (2011); Harmsen et al. (2010); and Chaturvedi et al. (2013). Concentrated hydrolysate is optionally prepared from desalted or otherwise “conditioned” solution derived from hydrolysis of pretreated material that was washed to remove extractives, using methods known in the art known and described in US20100151538 and US20110318798.

### EXAMPLE 6

### Production of Recombinant Algal Biomass in Wood-Derived Lignocellulosic Feedstock

This non-limiting example is directed to a recombinant algal cell that is cultured by the methods of the invention. In one embodiment, Chlamydomonas is cultured with a preferred culture medium comprising a wood-derived organic acid and a wood-derived lignocellulosic simplified sugar. A second case provides for a different recombinant algal cell of Scenedesmus that is cultured with a preferred culture medium comprising wood-derived lignocellulosic simplified sugar in the presence of a process residual of wood lignocellulose hydrolysis.

Heterotrophically adapted transgenic algae are maintained in 250 mL volumes in 500 mL flasks on an orbital shaker at 100 rpm at 28° C., initial pH of 7.0 as for Example 5 except urea is replaced with NH₄Cl for KAS1003. Both species of transgenic algae carry pChlamy_2 that contains the Aph7 (hygromycin resistance) gene under control of beta-tubulin promoter in their nucleus. Inoculum for the fermentor uses cells that are pelleted and re-suspended in wood-derived concentrates standardized to hydrocarbon. Fermentation proceeds as described in Example 5 above, using the reactor conditions disclosed in Example 3 of U.S. Ser. No. 62/356,896 except pure carbons sources are replaced with wood hydrolysates. Also the pH in KAS1003 fermentation is maintained at 7.4 with 0.5 M NaOH. Elucidation of transgene expression measures gene transcription by qRT-PCR. Primers designed using Primer3 (http://bioinfo.ut.ee/primer3-0.4.0/primer3/) monitor endogenous actin gene transcription compared to that of the transgene. Primers for actin in KAS1003 amplify 187 bp (5′-GATGCCCTGAGGTACTGTTC-3′ (SEQ ID NO: 1) and 5′-ACCTCCTTGCTCACTCTGTC-3′ (SEQ ID NO: 2)) and in KAS1001 amplify 163 bp (5′-ATAGCTTGCGTCTGGATAGG-3′ (SEQ ID NO: 3) and 5′-TGCGTCCTCTCATGTAAAAA-3′ (SEQ ID NO: 4)). Primers for Aph7 amplify a 111 bp (5′-CAACATCTTCGTGGACCTG-3″ (SEQ ID NO: 5) and 5′-AAGGCGTTGAGATGCAGT-3′ (SEQ ID NO: 6)). RNA is extracted from freeze dried biomass after 72 hr culture from dark-grown biomass at log phase (72 hrs) following manufacturer's instructions for RNeasy Plant Mini Kit (Qiagen 74903) and qRT-PCR performed following manufacturer's instructions for Superscript III One Step RT-PCR kit (Invitrogen 12574-018). The resulting qRT-PCR products quantification is compared relative to the non-transgenic control; KAS1003 has Aph7 expression 1.3 fold higher than actin and KAS1001 has Aph7 1.5 fold higher than actin. It is understood that a gene of interest can be further employed in transgenic algae as is known in the art using this expression system. For a target recombinant molecule at this volume of production at a mill-based biorefinery, these may be expressed compounds that confer animal or fish health as part of a whole biomass addition to the feed formulation, and further, may include those that accumulate the highest during the rapid biomass growth stage.

### EXAMPLE 7

### Additional Heterotrophic Co-Cultivation of Species in Wood-Derived Lignocellulosic Feedstcok

This example illustrates the potential of full utilization of sugars present in wood hydrolysates using a co-culture. Chlorella KAS908 and Rhodotorula KASI 101 are individually grown at 7-L fermentor scale in a medium containing model sugars to evaluate biomass growth and sugar utilization patterns on the major fixed carbons in Southern Hardwood Chips. With the previously determined fermentation conditions (see Example 1), KAS908 is grown in 2F-1+YE medium containing glucose and xylose at 19.43 g/L and 8.06 respectively. These sugar concentrations mimicked the sugar composition of Southern Hardwood Chips hydrolysate. KAS 1101 is grown in F/2+YE with the same glucose and xylose concentrations and fermentation conditions. Both strains show an increase in biomass productivity at different rates. KAS908 has a biomass productivity of 1.24 g/L/day and KAS1101 has a biomass productivity of 1.5 g/L/day. KAS908 and KAS1101 differ in the rates of glucose utilization, with the yeast depleting the glucose much more rapidly (FIG. 8). This suggested that, if starting with the same culture density in a co-culture, the yeast may outcompete the chlorophyte over time.

Dark shake flask experiments (heterotrophic) demonstrate the feasibility of biomass production by co-cultures of KAS908 (initial OD750=0.2) and KAS1101 (initial OD750 =0.2) on culture media with xylose/arabinose C5 sugars and glucose C6 sugar, or those sugars plus process residuals in a wood hydrolysate. Treatments are as follows, using cell cultures that are previously acclimated under heterotrophic conditions: a) F+YE+glucose (9 g/L); b) F+YE+glucose (4 g/L)+arabinose (2.5 g/L)+xylose (2.5 g/L); c) F+YE+arabinose (4.5 g/L)+xylose (4.5 g/L); and d) 30% SHC hydrolysate solution (equivalent to 9 g/L glucose) with F+YE.

All media treatments support growth of the co-cultures over a 6-day period. The co-culture on glucose alone stays green through day 6. The cultures grown in the media containing the glucose-arabinose-xylose blend or grown in the SHC hydrolysate are an orange-green by day 6, indicating the faster growth of the red yeast and its ability to utilize both C6 and C5 sugars present in the wood hydrolysate for growth. The co-culture grown in C5 arabinose-xylose sugars alone produces an eventual change to reddish-brown color similar to a KAS1101 monoculture, illustrating a faster growth of the Rhodotorula over the Chlorella on this substrate. To confirm that KAS1101 utilizes C5 sugars alone, a parallel growth experiment is carried out using F/2 medium containing only the C5 sugars: a) F/2+YE+arabinose (9 g/L); and b) F/2+YE+Xylose (9 g/L). Visual appearance of the flasks, with their more opaque cultures compared to the starting cultures, indicates that KAS1101 indeed utilizes C5 sugars (i.e., arabinose and xylose) for growth.

### EXAMPLE 8

### Assessment of Additional Species Performance in Wood-Derived Lignocellulosic Feedstock

Several algal species of commercial interest (Chlorella zofingiensis, Parachlorella spp., Scenedesmus obliquus and Mayamaea spp.) are tested for growth, pigments, and lipids on different wood hydrolysates at flask bioreactor level. This includes comparison with the model sugars and comparison of the different hydrolysate sources: Southern Hardwood Chips (SHC, 3% total sugar), Southern Pine Bleached Kraft (SPBK, 9% total sugar), Hardwood Chips (HWD, 4.4% total sugar) and Southern Pine Finer chips (SPFC, 2.15% total sugar). Chlorella zofingiensis KAS1170 (UTEX32) is grown in SHC, SPFC and HWD (normalized to 2 g/L total sugars C6+C5). Hawaiian Parachlorella KAS741 is grown in HWD (normalized to 2 g/L total sugars). Briefly, the wood hydrolysate solution is supplemented with F medium components and adjusted to pH 7.0 as per Example 1. Heterotrophically adapted KAS1170 is grown in F (as in Example 1) to log phase mixotrophically and photosynthetically on a 16/8 (day/night) cycle (i.e., to allow full depletion of residual glucose in the cultures before inoculating into wood hydrolysate-containing medium). To F medium containing the normalized 2 g/L sugar is added 0.2 g/L of yeast extract. To F medium containing wood hydrolysate normalized to 4 g/L is added 0.4 g/L yeast extract. All media are inoculated to an initial density of 1.0̂6 cells/mL. Cultures in shake flasks are allowed to grow for 7 days in the dark on an orbital shaker (INNOVA 4000 incubator shaker) at 28° C. and 120 rpm. Growth of KAS1170 (as dry weight) as well as glucose utilized are compared. Growth (DW) and pigment profile of KAS741 in HWD and model sugars are also compared. Biomass obtained is analyzed by TLC for pigment profiles using hexane:acetone (3:1) as running buffer on TLC plates (silica gel matrix, Sigma #Z122777)

Across the board, growth was observed in all wood hydrolysates using Chlorella zofingiensis and Parachlorella spp., with better growth from SPFC and HWD compared to model sugars. Among all the wood hydrolysates tested, KAS1170 grown in the softwood Southern Pine SPFC showed the highest biomass production with a 600-fold increase compared to the model sugars having a 400-fold increase (from Day 0) and a corresponding glucose utilization to biomass ratio of 1:0.81 (w/w) compared to 1:2.4 for the control on model sugars, indicating unexpected contributions to growth from the process residuals. Both KAS1170 and KAS741 grown on HWD also showed higher increase in biomass production than the model sugars, by 500-fold compared to less than 100-fold for KAS1170, and by 300-fold compared to 150-fold for KAS741 (from Day 0). KAS741 culture shows notable increase in viscosity from exopolysaccharide production, demonstrating that wood hydrolysates are suited to producing this phenotype and product. The exopolysaccharide can be separated from the cells and dried into a mass. Although KAS1170 had an increase in growth on SHC (88%) and SPBK (64%), there was a higher increase in biomass production on the model sugars of 1650% and 125%, respectively. KAS741 grown in hardwood HWD hydrolysate shows much lower pigmentation than on model sugars. KAS1170 shows lutein and astaxanthin contents varying among the different wood hydrolysates. KAS1170 grown in SHC hardwood hydrolysate has higher astaxanthin and lower lutein than model sugars. KAS1170 on softwood SPFC shows higher astaxanthin and lutein content than model sugars. KAS1170 on softwood SPBK and equivalent model sugars without process residuals have similar astaxanthin and lutein contents.

Scenedesmus obliquus KAS1003 and the diatom Mayamaea spp KAS1111 (See Kuehnle et al. 2015 for identification by DNA fingerprinting) are grown at 22° C. at 100 rpm in the dark in modified F medium containing 1.28 g/L glucose and 0.72 g/L xylose with nitrate replaced with equal molar NH₄Cl as the nitrogen source. A 25 mL of the log phase culture is used to inoculate 225 mL of control medium (same as previous) or to inoculate 225 mL (26.75 mL of hydrolysate labeled “Hardwood”, HWD, and 198.25 mL growth medium, pH adjusted to 7.0 with 1M Tris-HCl). Both the control medium and hydrolysate medium contained 1.28 g/L glucose and 0.72 g/L xylose at the start of the fermentation. On the third day a 100 mL sample was taken for analysis; the biomass was spun down at 3000 rpm for 5 minutes and freeze dried and the supernatant was collected for glucose analysis. On the fifth day another 100 mL of the remaining culture was collected for the same analysis performed on the third day of fermentation. Biomass was analyzed for pigments (TLC, spectrophotometer) and phospholipids (TLC). Total pigments and PLs were extracted from freeze dried ground biomass using 50 μL of chloroform:methanol (2:1) per mg of biomass. Debris was cleared by centrifugation at 8,000 rpm for 5 minutes. For pigments, absorbance at 470 nm was used to estimate differences in pigment content of crude extracts. For phospholipids, the 0.5 mg equivalent biomass was loaded onto a silica matrix gel (Sigma #Z233888) and were separated using chloroform:methanol:water (65:25:4) as the running buffer. Phospholipids were stained with molybdenum Blue spray reagent (Sigma M1942) and observed 20 minutes thereafter.

Results for KAS1003 showed 40% more biomass by day 3 (log phase) with glucose running out between day 3 and day 5. By day 5 (stationary phase, low glucose) the amount of biomass in each culture was equal but the hardwood HWD sample contained 1.5× more pigments than the control. Pigments from 0.2 mg equivalent biomass were separated by TLC run on silica gel matrix (Sigma #Z122777) using hexane:acetone (3:1) as running buffer, the bands for beta-carotene and lutein/zeaxanthin were observed in all samples, no detectable astaxanthin in any samples. We have demonstrated elsewhere that KAS1003 will generate astaxanthin in fermentation cultures once sufficient nutrient (N) deficiency occurs, and that did not occur in these 5 day old cultures in the treatment or controls. Once the specific growth rate is optimized for the KAS1003 using the preferred wood hydrolysate for a select pulp and paper mill, it is known in the art to time nitrogen feed during fed-batch fermentation for sufficient depletion to reach favorable specific productivity for a pigment such as astaxanthin. For lipids, measured as phospholipids content during growth phase before glucose depletion, there was no observable change on day 3 in the HWD and the control samples.

The diatom Mayamaea spp KAS1111 was grown and harvested in the same manner as above for KAS1003. Total pigments and phospholipids were extracted from freeze dried ground biomass using 50 μL of chloroform:methanol (2:1) per mg of biomass. Debris was cleared by centrifugation at 8,000 rpm for 5 minutes. Fucoxanthin from 0.2 mg of biomass was separated by TLC run on silica gel matrix (Sigma #Z122777) using hexane:acetone (3:1) as running buffer, the fucoxanthin band was cut out and eluted in acetone for absorbance at 470 nm readings. Fucoxanthin content was estimated on a dry weight basis by comparing to a dilution gradient of absorbance at 470 nm of commercially available fucoxanthin (Sigma F6932). Biomass generated was equal for both control and hardwood HWD samples for both day 3 and day 5. On day 3 (log phase) the control (0.55% pigment per DW) had 5× more fucoxanthin than the HWD (0.11% DW) sample; by day 5 (stationary phase, in which silica is lacking and glucose and ammonium were present) the control (0.63% pigment per DW) had 7× more fucoxanthin than the HWD (0.09% DW) sample. Unlike other carotenoids/xanthophylls, fucoxanthin is not a stress-induced pigment. Phospholipids content, monitored as a measure of lipids during the growth phase before glucose depletion, was similar on day 3 in the HWD and the control samples under the test conditions used.

Taken together, these results indicate the following advantages conferred using the method of the invention: Rapidly increased biomass yield, or reduced fermentation cycle time, using diluted hardwood and softwood hydrolysates; larger cells in some cases using hardwood hydrolysate when under higher osmotic pressure, useful for downstream processing; increased pigment yield in some hydrolysates for commercially valued pigments; and a strategy to use the method of the invention with added stress to generate additional desired pigment (astaxanthin). Further, using a diatom microalga with the method of the invention, the hardwood hydrolysate is seen to support growth (biomass production) very similar to the control medium lacking the lignocellulosic hydrolysis process residuals, and to significantly decrease the pigmentation of the biomass. This is advantageous for use of biomass in products where added color is unwanted.

### EXAMPLE 9

### Mixotrophic Cultivation and Product Formation Using Wood-Derived Lignocellulosic Hydrolysates

While these examples are largely exemplified for dark heterotrophic cultivation, it is understood that the methods of the invention can be applied without limitation to mixotrophic cultivation applicable for those species that are not obligate heterotrophs and are facultative heterotrophs. As a result of mixotrophy, productivity increases. This example is a modification of Example 5, such that heterotrophic growth of Haematococcus pluvialis KAS1601 is replaced by mixotrophic growth under 30 μE light. The growth rate of H. pluvialis KAS1601 is accelerated over dark culture such that a culture starting with 0.05 g/L biomass reaches an initial 9.0 g/L in 96 hours, with a pigment content of 2.4% DW. It is understood that higher productivities are obtainable with adjustment in feedstock and nutrition following mass balances, seed densities, aeration, and mixing, and light delivery, as is known in the art. When not used as whole-cell feed, pigment-extracted biomass as is known in the art is also suited as meal for fish, insect and animal feed applications, with the protein, beta-glucan, vitamins, micronutrients and residual pigment providing growth and health benefits.

### REFERENCES

1. Alvarez C, F M Reyes-Sosa, B Diez. 2016. Enzymatic hydrolysis of biomass from wood. Published online 2016 Feb. 1. doi: 10.1111/1751-7915.12346.

2. Ambati R R, Moi PS, Ravi S, and Aswthanarayana R G. 2014. Astaxanthin: Sources, extraction, stability, biological activities and its commercial application—a review. Marine Drugs. 12(1):128-152.

3. Bar E, Rise M, Vishkautsan M, and Arad S. 1995. Pigment and structural changes in Chlorella zofingiensis upon light and nitrogen stress. Journal of Plant Physiology 146:527-534.

4. Bumbak F, Cook S, Zachleder V, et al. 2011. Best practices in heterotrophic high cell density microalgal processes: achievements, potential, and possible limitations. Applied Microbiology Biotechnology 91:31-46.

5. Chandel A K, S S da Silva, O V Singh. 2011. Detoxification of Lignocellulosic Hydrolysates for Improved Bioethanol Production, Biofuel Production-Recent Developments and Prospects, Dr. Marco Aurelio Dos Santos Bernardes (Ed.), ISBN: 978-953-307-478-8, InTech, see world-wide website: intechopen.com/books/biofuel-production-recent-developments-and-prospects/detoxification-of-lignocellulosic-hydrolysates-for-improved-bioethanol- production.

6. Chen T, Wei D, Chen G, et al. 2009. Employment of organic acids to enhance astaxanthin formation in heterotrophic Chlorella zofingiensis. Journal of Food Processing and Preservation 33:271-284.

7. Chisti Y. 1999. Fermentation (Industrial): Basic Considerations. In: Robinson R, Batt C, and Patel P, eds. Encyclopedia of Food Microbiology. Academic Press, London, pp 663-674.

8. Chua, PR, Franklin S, Wee J, Desai R. 2011. Production of Soladiesel® from cellulosic feedstocks. Energy Research and Development Division Final Project Report, California Energy Commission.

9. Doebbe A, Rupprecht J, Beckman J, et al. 2007. Functional integration of the HUP1 hexose symporter gene into the genome of C. reinhardtii: Impacts on biological H₂ production. Journal of Biotechnology 131:27-33.

10. Hawkins, R L. 1999. Utilization of xylose for growth by the eukaryotic algae, Chlorella. Current Microbiology 38:360-363.

11. Jonsson L J and C. Martin. 2016. Pretreatment of lignocellulose: Formation of inhibitory by-products and strategies for minimizing their effects. Bioresource Technology 199: 103-112.

12. Kot A M, S Blażejak, A Kurcz, I Gientka, M Kieliszek. 2016. Rhodotorula glutinis—potential source of lipids, carotenoids, and enzymes for use in industries. Appl Microbiol Biotechnol. 100: 6103-6117.

13. Kumar A, D M Barrett, M J Delwiche, and P Stroeve. 2009. Methods for Pretreatment of Lignocellulosic Biomass for Efficient Hydrolysis and Biofuel Production Ind. Eng. Chem, Res., Article ASAP.DOI: 10.1021/ie801542g.

14. Lee Y K and Zhang D H. 1999. Production of astaxanthin by Haematococcus. Chemicals from Microalgae 41-56.

15. Mohagheghi A, M Ruth, D J Schell. 2006. Conditioning hemicellulose hydrolysates for fermentation: Effects of overliming pH on sugar and ethanol yields. Process Biochemistry 41: 1806-1811.

16. Mao H, J M Genco, A van Heiningen, H Pendse. 2010. Kraft mill biorefinery to produce acetic acid and ethanol: Technical economic analysis. BioResources 5(2): 525-544.

17. Morales-Sanchez D, O A Martinez-Rodriguez, J Kyndt, and A Martinez. 2015. Heterotrophic growth of microalgae: metabolic aspects. World J Microbiol Biotechnol 31:1-9.

18. Normark M, S Winestrand, T A Lestander, and L J Jönsson. 2014. Analysis, pretreatment and enzymatic saccharification of different fractions of Scots pine. BMC Biotechnol 14:20 Published online 2014 Mar. 19. doi: 10.1186/1472-6750-14-20.

19. Olstorpe M, A Vidakovic, D Huyben, A Kiessling. 2014. A technical report on the production of microbial protein. Aquabest, Finnish Game and Fisheries Research Institute, Helsinki, 2014, ISBN 978-952-303-088-6.

20. Palmqvist E, Hahn-Hagerdal B. 2000. Fermentation of lignocellulosic hydrolysates I: inhibition and detoxification. Bioresource Technol 74:17-24.

21. Perez M, N A Nolasco, A Vasavada, M Johnson, A Kuehnle. 2015. Algae-mediated valorization of industrial waste streams. Ind Biotech 11(4): 229-234.

22. Perez-Garcia O, FME Escalante, L E de-Bashan, Y Bashan. 2011. Heterotrophic cultures of microalgae: Metabolism and potential products. Water Research 45:11-36.

23. Pienkos P T, M Zhang. 2009. Role of pretreatment and conditioning processes on toxicity of lignocellulosic biomass hydrolysates. Cellulose 16: 743-762.

24. Pirastru L, M Darwish, F L Chu, F Perreault, L Sirois, L Sleno, R Popovic. 2012. Carotenoid production and change of photosynthetic functions in Scenedesmus sp. exposed to nitrogen limitation and acetate treatment. J Appl Phycol 24:117-124.

25. Rana V., A. D Eckard, and B. K. Ahring 2014. Comparison of SHF and SSF of wet exploded corn stover and loblolly pine using in-house enzymes produced from T. reesei RUT C30 and A. saccharolyticus.

26. Ren L J, Huang H, Xia A H, et al. 2009. Enhanced docosahexaenoic acid production by reinforcing acetyl-CoA and NADPH supply in Schizochytrium sp. HX-308. Bioprocess Biosyst Eng. 32:837-843.

27. Rise M, Cohen E, Vishkautsan M, et al. 1994. Accumulation of secondary carotenoids in Chlorella zofingiensis. Journal of Plant Physiology 144:287-292.

28. Scaife M A, Nguyen G T D T, Rico J, et al. 2015. Establishing Chlamydomonas reinhardtii as an industrial biotechnology host. The Plant Journal 82:532-546.

29. Scranton M A, J T Olstrand, F J Fields, S P Mayfield. 2015. Chlamydomonas as a model for biofuels and bio-products production. Plant Journal 82: 523-531.

30. Su Y, Wang J, Shi M, Niu X, Yu X, Gao L, Zhang X, Chen L, Zhang W. 2014. Metabolomic and network analysis of astaxanthin-producing Haematococcus pluvialis under various stress conditions. Bioresource Technol. 170:522-9.

31. Towers L. 2015. Feeding the future salmon industry with trees. Sustainable Aquaculture Digital, pp 16-20.

32. USDA. 1971. Bark and its possible uses. Madison, Wis., US Department of Agriculture.

33. Wang Y and Peng J. 2008. Growth-associated biosynthesis of astaxanthin in heterotrophic Chlorella zofingiensis (Chlorophyta). World Journal of Microbiology and Biotechnology 24 (9): 1915-1922.

34. White E M. 2010. Woody biomass for bioenergy and biofuels in the United States—a briefing paper. USDA Forest Service General Technical Report PNW-GTR-825 45 pp. JAKAS\108X\Application\subspec-clean5.docx/mep

35. Zha Y, J A Westerhus, B Muilwijk et al. 2014. Identifying inhibitory compounds in lignocellulosic biomass hydrolysates using an exometabolomics approach. BMC Biotechnolog 14:22. See world-wide website: biomedcentral.com/1472-6750/14/22.

36. Chaturvedi V, P Verma. 2013. An overview of key pretreatment processes employed for bioconversion of lignocellulosic biomass into biofuels and value added products. 3 Biotech 3:415-431.

37. Harmsen P F H, W J J Huijgen, L M Bermúdez López, R R C Bakker. 2010. Literature Review of Physical and Chemical Pretreatment Processes for Lignocellulosic Biomass. BioSynergy project (2007-2010). Wageningen University ECN-E-10-013.

38. Brodeur G, E Yau, K Badal, J Collier, K B Ramachandran, and S Ramakrishnan. 2011. Chemical and Physicochemical Pretreatment of Lignocellulosic Biomass: A Review. Enzyme Research Volume 2011, Article ID 787532, 17 pages doi:10.4061/2011/787532

39. Burkhardt S, L Kumar, R Chandra and J Saddler 2013. How effective are traditional methods of compositional analysis in providing an accurate material balance for a range of softwood derived residues? Biotechnology for Biofuels 6:90. See, for example, world-wide website: biotechnologyforbiofuels.com/content/6/1/90

What is claimed is:

1. A method of producing a culture medium, the method comprising:
a) providing a lignocellulosic biomass, wherein the lignocellulosic biomass comprises a lignocellulosic compound,
b) hydrolyzing the lignocellulosic biomass to produce a lignocellulosic hydrolysate, wherein the lignocellulosic hydrolysate comprises a simplified sugar produced from at least a portion of the lignocellulosic compound,
c) optionally, separating the lignocellulosic hydrolysate into a first portion and a second portion and treating the second portion of the lignocellulosic hydrolysate to convert a portion of the lignocellulosic compound and/or the simplified sugar to a non-sugar agent;
d) optionally, mixing the treated second portion of the lignocellulosic hydrolysate comprising the non-sugar agent with the first portion of the lignocellulosic hydrolysate,
e) producing a culture medium comprising the lignocellulosic hydrolysate obtained after step b) or comprising the mixture obtained after steps c) and d).

2. The method of claim 1, wherein the non-sugar agent is an organic acid, an alcohol, a micronutrient, a salt, a saponifiable or fatty acid compound, a furfural, a process water, a protein, or any combination thereof.

3. The method of claim 1, wherein the step of treating the second portion of the lignocellulosic hydrolysate comprises culturing the second portion with a microbe that converts the portion of the lignocellulosic compound to the non-sugar agent.

4. The method of claim 2, wherein the organic acid is acetic acid, propionic acid, citric acid, fumaric acid, glycolic acid, lactic acid, malic acid, pyruvic acid, succinic acid, glucuronic acid, galacturonic acid, ferulic acid or any combination thereof.

5. A culture medium produced according to the method of claim 1.

6. A method for synthesizing a product of interest, the method comprising:
providing the culture medium of claim 5;
providing a microalgal cell that produces the product of interest;
culturing the microalgal cell in the culture medium to produce a microalgal culture from the microalgal cell; and
purifying the product of interest from the microalgal cells.

7. The method of claim 6, where the step of culturing the microalgal cell is performed in the dark.

8. The method of claim 6, wherein the product of interest is altered in component composition, proportion, or temporal expression as compared to a control, wherein said control is a product of interest produced by culturing a microalgal cell expressing said product of interest in culture medium comprising the simplified sugar but not containing the non-sugar agent.

9. The method of claim 6, wherein the product of interest is a microalgal biomass comprising the microalgal cells.

10. The method of claim 6, wherein the product of interest is a protein, lipid, polysaccharide, pigment, recombinant molecule, a functional component thereof, or any combination thereof.

11. The method of claim 10, wherein the lipid is a polyunsaturated fatty acid, a triglyceride, a phospholipid, or any combination thereof.

12. The method of claim 11, wherein the polyunsaturated fatty acid is an omega-3 fatty acid, omega-6 fatty acid, omega-9 fatty acid, DHA, EPA, linolenic acid, linoleic acid, or any combination thereof.

13. The method of claim 10, wherein the pigment is astaxanthin or lutein or a combination thereof.

14. The method of claim 6, wherein the microalgal cell is capable of utilizing a hexose or pentose sugar.

15. The method of claim 6, wherein the microalgal cell is a facultative heterotroph, capable of mixotrophy or heterotrophy, or an obligate heterotroph.

16. The method of claim 6, wherein the growth and synthesis of the product of interest occurs during the step of culturing, and wherein the step of culturing is performed under a fed-batch fermentation.

17. The method of claim 6, wherein the synthesis of the product of interest occurs under nutrient replete or nutrient deplete conditions.

18. The method of claim 6, wherein the simplified sugar is a hexose or pentose monomeric sugar.

19. The method of claim 6, wherein the non-sugar agent is a process residual of wood lignocellulose hydrolysis comprising an organic acid, an alcohol, a micronutrient, a salt, a saponifiable or fatty acid compound, a furfural, a process water, a protein, or any combination thereof.

20. The method of claim 6, wherein the wood lignocellulosic simplified sugar is an unconverted residual present in a microbial fermentation solution.

21. The method of claim 6, wherein the culture medium comprises a sugar to nitrogen ratio equilibrated to allow accumulation of the product of interest.

22. The method of claim 6, with a bioconversion ratio of 0.45:1 or higher, preferably of 1:1 or higher units of biomass produced per unit of sugar utilized.

23. The method of claim 6, wherein the method further comprises one or more steps of drying, grinding, lysing, or extracting the microalgal cell.

24. The method of claim 6, wherein the microalgal cell is a first cell type that is cultivated with a different second cell type.

25. The method of claim 24, wherein the second cell assimilates the same or a different fixed carbon source as the first cell.

26. The method of claim 6, wherein the cell belongs to class Chlorophyceae, Bacillariophyceae, Trebouxiophyceae, Euglenophyceae, Peridinea, Dinophyceae, or Labyrinthulomycetes.

27. The method of claim 24, wherein the second cell is a Rhodotorula.

28. A method of processing a lignocellulosic biomass, the lignocellulosic biomass comprising a lignocellulosic compound, the method comprising the steps of:
a) providing the lignocellulosic biomass;
b) hydrolyzing the lignocellulosic biomass to produce a lignocellulosic hydrolysate, wherein the lignocellulosic hydrolysate comprises a simplified sugar produced from at least a portion of the lignocellulosic compound;
c) optionally, separating the lignocellulosic hydrolysate into a first portion and a second portion and treating the second portion of the lignocellulosic hydrolysate to convert a portion of the lignocellulosic compound and/or the simplified sugar to a non-sugar agent; and
d) optionally, mixing the treated second portion of the lignocellulosic hydrolysate comprising the non-sugar agent with the first portion of the lignocellulosic hydrolysate.

29. The method of claim 28, wherein the non-sugar agent is an organic acid, an alcohol, a micronutrient, a salt, a saponifiable or fatty acid compound, a furfural, a process water, a protein, or any combination thereof.

30. The method of claim 28, wherein the step of treating the second portion of the lignocellulosic hydrolysate comprises culturing the second portion with a microbe that converts the portion of the lignocellulosic compound to the non-sugar agent.

31. The method of claim 29, wherein the organic acid is acetic acid, propionic acid, citric acid, fumaric acid, glycolic acid, lactic acid, malic acid, pyruvic acid, succinic acid, glucuronic acid, galacturonic acid, or ferulic acid.

32. The method of claim 3, wherein the microbe utilizes the organic acid as the sole carbon source for growth.

33. The method of claim 26, wherein the cell belongs to the class Chlorophyceae or Dinophyceae.

34. The method of claim 6, wherein the product of interest is altered in component composition, proportion, or temporal expression as compared to a control, wherein said control is a product of interest produced by culturing a microalgal or microbial cell expressing said product of interest in culture medium comprising non-lignocellulosic sugars.

35. The method of claim 10, wherein the recombinant molecule is a double-stranded RNA or a protein.

36. The method of claim 10, wherein the polysaccharide is a beta-glucan or an exopolysaccharide.

37. The method of claim 30, wherein the microbe u the organic acid as the sole carbon source for growth.
